SEQUENCE LISTING (12 sequences)

SEQ ID NO: 1 - DNA, 19 bp, synthetic construct
  source 1..19: mol_type = other DNA
tgacactggcaaaacaatg

SEQ ID NO: 2 - DNA, 20 bp, synthetic construct
  source 1..20: mol_type = other DNA
ggcttatatccaacacttcg

SEQ ID NO: 3 - DNA, 18 bp, synthetic construct
  source 1..18: mol_type = other DNA
cctgagtttcaagtatcc

SEQ ID NO: 4 - DNA, 18 bp, synthetic construct
  source 1..18: mol_type = other DNA
aacaacagaacacaacaa

SEQ ID NO: 5 - DNA, 18 bp, synthetic construct
  source 1..18: mol_type = other DNA
gagtctgttctggcttat

SEQ ID NO: 6 - DNA, 19 bp, synthetic construct
  source 1..19: mol_type = other DNA
aactggaatatcacaaggt

SEQ ID NO: 7 - DNA, 19 bp, synthetic construct
  source 1..19: mol_type = other DNA
gccgctcgtcagactccag

SEQ ID NO: 8 - DNA, 18 bp, synthetic construct
  source 1..18: mol_type = other DNA
gcagcgtccaaggcttcc

SEQ ID NO: 9 - DNA, 18 bp, synthetic construct
  source 1..18: mol_type = other DNA
tggcttgtggtgtaattg

SEQ ID NO: 10 - DNA, 20 bp, synthetic construct
  source 1..20: mol_type = other DNA
tagcaggataacagatgagt

SEQ ID NO: 11 - DNA, 18 bp, synthetic construct
  source 1..18: mol_type = other DNA
ggaggcaagaccttcatt

SEQ ID NO: 12 - DNA, 18 bp, synthetic construct
  source 1..18: mol_type = other DNA
atggatcagcagcaagtc

(19) United States
(12) Patent Application Publication
(10) Pub. No.: US 2026/0263401 A1
(43) Pub. Date: Sep. 10, 2026

(54) Topical composition and uses thereof

(71) Applicants: UNIVERSIDADE DE COIMBRA, Coimbra (PT); TOXFINDER, LDA., Coimbra (PT)

(72) Inventors: Maria Teresa DE TEIXEIRA CRUZ, Coimbra (PT); Gonçalo BRITES, Coimbra (PT); Isabel FERREIRA DO VALE, Coimbra (PT); Ana Cristina ROSA SILVA, Coimbra (PT); Carla Sofia PINHEIRO VITORINO, Coimbra (PT); Maria Margarida MARTINS GONÇALO, Coimbra (PT); Bruno Miguel RODRIGUES DAS NEVES, Coimbra (PT)

(73) Assignees: UNIVERSIDADE DE COIMBRA, Coimbra (PT); TOXFINDER, LDA., Coimbra (PT)

(21) Appl. No.: 18/994,870

(22) PCT Filed: Jul. 14, 2023
(86) PCT No.: PCT/IB2023/057222

§ 371 Date: Jan. 15, 2025

(30) Foreign Application Priority Data
Jul. 15, 2022 (PT) 118109

(51) Int. Cl. A61K 31/198; A61K 9/00; A61K 38/02; A61K 47/10; A61K 47/12; A61K 47/32; A61P 17/04

(52) U.S. Cl. CPC A61K 31/198; A61K 9/0014; A61K 38/02; A61K 47/10; A61K 47/12; A61K 47/32; A61P 17/04

(57) ABSTRACT

The present disclosure relates to a hydrogel for use in the prevention, treatment or therapy of skin allergies comprising an allergen scavenger, wherein said allergen scavenger is a mixture of two amino acid selected from n-acetyl cysteine, lysine, cysteine or their pharmaceutical acceptable salts, or a mixture of two peptide comprising a terminal amino acid selected from n-acetyl cysteine, lysine, cysteine or their pharmaceutical acceptable salts, or mixtures thereof. Furthermore, the present disclosure also relates to topical compositions comprising such hydrogel, and the use in several skin allergies, namely in the prevention, treatment, or therapy of contact dermatitis.

### TECHNICAL FIELD

The present disclosure relates to a hydrogel for use in the prevention, treatment or therapy of skin allergies comprising an allergen scavenger, wherein said allergen scavenger is a mixture of two amino acid selected from n-acetyl cysteine, lysine, cysteine or their pharmaceutical acceptable salts, or a mixture of two peptide comprising a terminal amino acid selected from n-acetyl cysteine, lysine, cysteine or their pharmaceutical acceptable salts, or mixtures thereof. These compounds are able to chemically sequester skin allergens thus avoiding their interaction with skin proteins and, consequently, inhibiting the formation of the immunogenic complexes responsible for the early cellular and molecular mechanisms triggering the development of dermatitis, namely allergic contact dermatitis (ACD). The present disclosure also relates to topical compositions comprising such hydrogel, and the use in several skin allergies, namely in the prevention, treatment or therapy of contact dermatitis.

### BACKGROUND

Skin problems related to contact with chemicals are a continuous growing environmental and occupational health problem. Two main types of contact dermatitis are described considering the pathophysiological mechanism involved: irritant contact dermatitis (ICD), and allergic contact dermatitis (ACD) [1,2]. Irritant contact dermatitis is the most common occupational skin disease, accounting for 80% of all contact dermatitis cases. It can occur after a single exposure to an irritant or toxic substance (e.g. abrasives, cleaning, oxidizing, and reducing agents) and is characterized by skin damage resulting from a direct and local cytotoxic effect on the skin's cells [3].

ACD is an adverse cutaneous inflammatory reaction caused by the direct contact of the skin with a specific exogenous allergen to which humans have previously been sensitized [4]. ACD has a significant impact on healthcare costs and industry productivity due to the absence of work [5]. For instance, in Europe, it is estimated that the annual incidence of occupational contact dermatitis ranges from 0.5 to 1 case per 1000 workers [6]. Furthermore, ACD represents the principal and most prevalent form of immunotoxicity found in humans [7,8] and accounts for 30% of all cases of occupational diseases in industrialized nations [4] and 4-7% of all dermatological consultations [9].

The prevalence of ACD has been increasing worldwide for the past few decades [10], currently affecting 15-20% of the general population worldwide with 5-10% developing clinical symptoms at least once a year [1]. After multiple exposures to the sensitizing agent, individuals develop the clinical manifestations, often characterized by intense pruritus, stinging and pain accompanied by well-demarcated erythema and oedema [3].

Many occupational activities such as hairdressers and beauty and wellness professionals are prone to develop this skin condition. Indeed, ACD is a multifactorial disease in which genetic and environmental factors play a role. Genetic predisposition involves polymorphisms in genes that regulate xenobiotic metabolism and biotransformation, redox balance, inflammation, and skin barrier functions [11].

The most relevant risk factors include exposure to the workplace, age, and sex (most frequent in young females) [11,12]. From a pathophysiological point of view, ACD is a type IV hypersensitivity response caused by low molecular weight (LMW) reactive chemicals (<1000 Da, also called haptens) and metal ions such as nickel [Ni]²⁺ and chromium [Cr]³⁺ [13]. It is characterized by two distinct phases, involving both innate and adaptive immune responses. First, the sensitization phase also referred to as the afferent or induction phase includes the events following the first contact with the allergen, in which the sensitizing chemical penetrates the stratum corneum, driving the development of effector T cells. Second, the elicitation phase also referred to as the effector or challenge phase, where sensitized individuals are re-exposed to the sensitizing chemical, leading to clinical manifestation, usually within 24/72 h [11,14].

During the sensitization phase, LMW chemicals or haptens endowed with electrophilic properties react with an endogenous protein, rendering it immunogenic [12,15,16]. Additionally, skin allergens also activate the signaling pathway nuclear factor erythroid 2-related factor 2 (Nrf2), which in turn is translocated to the nucleus inducing the transcription of over 100 protective genes [17]. Then, these protein conjugates induce stress responses and xenoinflammation through the release of damage-associated molecular patterns (DAMPs), such as reactive oxygen species (ROS), uric acid, hyaluronic acid fragments and ATP/ADP [18]. These DAMPs activate intracellular signaling pathways in antigen presenting cells such as dendritic cells (DCs), leading to their maturation. Subsequently, DCs process the conjugates and migrate to the draining lymph nodes where they prime naive T lymphocytes. T-cells become activated and expand into allergen-specific effector T-cells that disseminate systemically and elicit a strong inflammatory reaction upon later contact with the same chemical leading to a gradually developing eczematous reaction [11].

Currently, there are more than 4000 substances known as potential chemical sensitizers [19,20], and every year, several new contact allergens are identified, constantly expanding the horizon of known causes of ACD [21]. Recently, impurities from the synthesis process of several compounds used in consumer products have been described as the actual sensitizers [22]. Common skin allergens include transition metals (nickel, chromium, and cobalt), fragrances, preservatives, multiple topical drugs, hair dyes, acrylates, rubber chemicals, epoxy resins, and plant defense-related substances [22].

Currently, the definitive treatment of ACD relies on the identification and subsequent removal of any potential causal agent, otherwise, there's an increased risk for chronic or recurrent dermatitis [23].

A relevant problem of ACD is the absence of a cure and once an individual is sensitized he develops clinical manifestations upon re-exposure to the same chemical. Therefore, most guidelines recommend ceasing exposure to the triggering chemical and there's a need to develop preventive strategies such as the use of hand and body protection approaches. The current great challenge is to develop topical preparations that effectively prevent the development of ACD.

Until now, and focused on ACD preventive purposes, the industry has latex gloves, barrier creams, emollients and moisturizers. However, studies on using gloves to prevent the development of occupational contact dermatitis are limited and the observed benefits were not exclusively attributable to wearing gloves, because they were only one part of a comprehensive prevention strategy.

Concerning barrier creams, water resistant barrier creams contain hydrophobic substances such as silicone, which protects against water soluble substances such as acids, alkali and dye. On the other hand, oil or solvent resistant barrier creams protect against dust, oils, greases and solvents, but their clinical effectiveness in preventing ACD is controversial and unsupported by clinical studies. Furthermore, barrier creams should be used on normal skin as they cause aggravation of dermatitis if applied to inflamed skin [24,25].

Among the ingredients used in topical formulations, there is robust evidence that the topical skin protectant quaternium-18-bentonite can prevent urushiol-induced ACD and that the chelating agent diethylenetriaminepentaacetic can prevent nickel, chromium, and copper dermatitis [26].

The product NIK-L-BLOK® claims to have chelating effects over nickel. Indeed, NIK-L-BLOK® is a skin barrier cream that uses the active ingredient diethylenetriamine pentaacetic acid to capture free nickel ions, thus blocking their permeation through the skin. The cream's ingredients work effectively to protect the skin, preventing allergic reactions towards nickel, such as eczema, dryness, blisters, redness and itching [27].

Skintifique®, another barrier cream available is marketed as an extremely safe and pure composition to be used to prevent skin rash by scavenging metals like nickel, chromium and cobalt at the skin surface. The patented method called “the capturifique technology” is not described in detail but claimed to capture particles of pollution and metals at the surface of the skin preventing them from penetrating the epidermis. The protective effect of one application is said to last up to 14 hours, but the cream must be reapplied after each washing of the skin [28]. The manufacturers also claimed that in more than 70% of the conducted tests the volunteers did not experience any adverse skin reaction upon exposure to the objects.

Interestingly, the Bariéderm® cream, usually used as a repairing cream is currently on clinical trials for evaluation of the efficiency and tolerability in protecting individuals with hand dermatitis, either due to allergy or irritation. On this trial, with the primary propose of treatment, the patients are asked to apply the barrier cream twice a day on their hands for 3 weeks. Although barrier creams are usually recommended for use before work and at least two to three times during work time, the optimal frequency of application remains to be clearly defined [25,26].

Overall, it is important to emphasize that there is a very small body of evidence, with a few good quality studies, that pre-work or barrier creams improve clinical indicators of skin condition [25,26].

Moisturizers and emollients used alone or in combination with barrier creams may result in a clinically important protective effect, either in the long- or short-term, for the primary prevention of ACD. Moisturizers improve the hydration of the stratum corneum by the use of humectants such as glycerin, urea, sorbitol, pyrrolidone carboxylic acid, and the emollients petrolatum, lanolin, mineral oil, silicone, and waxes. They are also used for regenerative skincare, during and after work, and should be applied regularly during work time after hand washing and after work at home to support the regenerative capacities of the skin [29-31]. Regardless of their protective role in preventing chemical absorption, in some cases, emollients may act as conduits for chemicals. Indeed, some ingredients present in emollients, can also act as haptens, mainly preservatives (e.g. imidazolidinyl urea, formaldehyde and methylisothiazolinone) and fragrances but also emulsifiers and humectants [32-34]. Therefore, primary care practitioners should consider the excipients in emollients, which can cause new onset rashes or aggravate existing skin conditions.

WO2013138744A1 describes compositions comprising a fluorinated corticosteroid and N-acetylcysteine (NAC) for use in several disease disorders, where increased or abnormal disulfide bonds are found. In this document the use of NAC is related with its mechanism on converting oxidized disulfide bonds between proteins to reduced, free thiol groups, leaving the proteins dissociated, helping to shed thickened layers of skin.

WO2020146666A1 describes a method for the use of N-acetylcysteine (NAC), N-acetylcysteine amide (NACA) or (2R,2R′)-3,3′-disulfanediyl bis(2-acetamidopropanamide) (diNACA) for the prevention and/or treatment of at least one of: radiation dermatitis, radiation damage to the skin, skin lightening, whitening or to improve skin health in an animal or human. The document provides a method for the prevention, amelioration, or treatment of a disease or condition associated with oxidative stress in a subject comprising administration of a therapeutically effective amount of NACA, to increase the amount of glutathione expressed in the tissues of the subject.

EP0957927B1 describes a composition for use as a medicament in a topical barrier formulation, characterised in that it comprises a cationic, hydrophilic, amine containing polymer; bound to an anionic scavenger substance, which is a) either chosen from the group consisting of anionic ethylene amine compounds, tetraazacykloalkane-N,N,N,N-tetraacetic acids; and polymer derivatives of porphyrines, or b) is an endogenous compound, chosen from the group consisting of taurine, hypotaurine and chlorinated or carbamylated derivatives thereof, cysteamine, cysteine, N-acetylcysteine and acidic metabolites of polyamines, with the proviso that native chitosan or derivatives thereof is/are not covalently bound to DTPA or EDTA.

Taking the above into consideration, namely the lack of effective strategies for the prevention of ACD against the number of increasing skin sensitizers to which humankind are exposed, in addition to metals, and focused on the market demand, the present disclosure relates to a novel topical composition enriched in molecules capable of chemically sequestering skin allergens thus avoiding its interaction with skin proteins (the first adverse outcome pathway for skin sensitization) and consequently preventing the development of allergic contact dermatitis. Of utmost importance, the mechanism of action of barrier creams and all the ingredients above listed is quite different from the molecules presented in this invention, which constitutes the innovative character of this topical composition.

These facts are disclosed in order to illustrate the technical problem addressed by the present disclosure.

### GENERAL DESCRIPTION

The present disclosure relates to a hydrogel for use in the prevention, treatment or therapy of skin allergies comprising an allergen scavenger, wherein said allergen scavenger is a mixture of two amino acid selected from n-acetyl cysteine, lysine, cysteine or their pharmaceutical acceptable salts, or a mixture of two peptide comprising a terminal amino acid selected from n-acetyl cysteine, lysine, cysteine or their pharmaceutical acceptable salts, or mixtures thereof. Furthermore, the present disclosure also relates to topical compositions comprising such hydrogel, and the use in several skin allergies, namely in the prevention, treatment or therapy of contact dermatitis.

A skin allergy is when skin becomes irritated because the immune system reacts to something that is usually harmless. An allergic reaction can cause rash, itching, burning, redness, bumps, hives, and swelling. Many different allergens can cause a reaction. Below are some of the most common allergic skin conditions:
Atopic dermatitis or eczema is when the skin becomes easily irritated, itchy, and dry. It is the most common allergic skin condition and is more common in children than adults. Eczema is linked to both genetic (inherited from parents) and environmental factors. It is connected to asthma, food allergies, and seasonal allergies. Some things can make eczema flare up, such as certain foods, stress, soaps and lotions, or cold and dry air.
Allergic contact dermatitis is when something touches the skin and causes a reaction. For example, some people are allergic to the metal nickel and will have a skin reaction if jewelry made with nickel touches their skin. A reaction to poison ivy is another example. Many people have an allergic reaction to the oil on a poison ivy or poison oak plant.
Urticaria or hives, are raised bumps on the skin that form because of an allergic reaction. These bumps are also called welts or wheals. A person may get hives after eating a food they're allergic to. The bumps are a result of histamine that the body releases in response to the allergen. Things besides allergies can cause hives as well, such as a bug bite.
Angioedema is swelling deep in the skin. It often happens in places like the eyelids, lips, and throat, and often happens together with hives.

An aspect of the present disclosure relates to a hydrogel for use in the prevention, treatment or therapy of skin allergies comprising an allergen scavenger, wherein said allergen scavenger is:
a mixture of two amino acid selected from a list consisting of: n-acetyl cysteine, lysine, cysteine or their pharmaceutical acceptable salts; or
a mixture of two peptide comprising a terminal amino acid selected from a list consisting of: n-acetyl cysteine, lysine, cysteine or their pharmaceutical acceptable salts, or mixtures thereof.

In an embodiment, the concentration of the first amino acid or peptide ranges from 0.2% to 2.0% (w/w), preferably 0.16% to 1.6% (w/w); and the concentration of the second amino acid or peptide ranges from 0.2% to 2.0% (w/w), preferably 0.15% to 1.5% (w/w).

In an embodiment, the hydrogel comprises n-acetyl cysteine and lysine, or their pharmaceutical acceptable salts, as allergen scavengers.

In an embodiment, the concentration of n-acetyl cysteine or their pharmaceutical acceptable salt ranges from 0.2% to 2.0% (w/w), and the concentration of lysine or their pharmaceutical acceptable salt ranges from 0.2% to 2.0% (w/w).

In an embodiment, the hydrogel is selected from a list consisting of: carbomer (Carbopol); chitosan, gelatin, alginate, laminarin, hyaluronic acid, poly(vinyl alcohol), polyacrylamide, carboxymethylcellulose, sodium starch glycolate, sodium carboxymethyl starch, dextran, dextran sulfate, xanthan, gellan, pectinic acid, deoxyribonucleic acids, ribonucleic acid, albumin, polyacrolein potassium, sodium glycine carbonate, poly(acrylic acid) and its salts, polyacrylonitrile, poly(styrene sulfonate), poly(aspartic acid), polylysine, polyvinylpyrrolidone, polyvinyl alcohol, ultramylopectin, poly(ethylene glycol), neutral cellulose derivatives, microcrystalline cellulose, powdered cellulose, cellulose fibers, or starch; or combinations; preferably carbomer (Carbopol).

In an embodiment, the hydrogel further comprises keratin, collagen, glutathione, homocysteine, 2,3 dimercaptosuccinic acid, methionine, bovine serum albumin, or mixtures thereof.

In an embodiment, the hydrogel further comprises a chelating agent; preferably NA₂EDTA.

In an embodiment, the hydrogel further comprises a steroidal anti-inflammatory agent, a non-steroidal anti-inflammatory agent, a histamine H1-receptor antagonist, or mixtures thereof.

In an embodiment, the hydrogel comprises
n-acetyl cysteine, in a concentration ranging from 0.2 to 2.0% (w/w); preferably 1.6% (w/w),
lysine, in a concentration ranging from 0.2 to 2.0% (w/w); preferably 1.5% (w/w), Na₂EDTA, in a concentration ranging from 0.01 to 0.1% (w/w); preferably 0.1% (w/w),
benzoic acid, in a concentration equal or below 0.1% (w/w); preferably 0.1% (w/w), Carbomer Interpolymer Type A, in a concentration ranging from 0.5 to 2.0% (w/w); preferably 2.0% (w/w),
isopropanol, in a concentration equal or below 30.0% (w/w); preferably 30% (w/w), glycerol, in a concentration equal or below 10.0% (w/w); preferably 10% (w/w), water, in a concentration equal or below 70.00% (w/w); preferably 56% (w/w).

Another aspect of the present disclosure relates to a topical composition comprising said hydrogel and acceptable pharmaceutical excipients.

In an embodiment, the topical composition is selected from a list consisting of: solution, serum, cream, lotion, gel, hydrogel, oil, soap, shampoo, stick- or bar-shaped solid, spray, ointment, paste, mousse or body wash.

Another aspect of the present disclosure relates to the hydrogel or the topical composition for use in the prevention, treatment or therapy of adverse cutaneous inflammatory reaction.

Another aspect of the present disclosure relates to the hydrogel or the topical composition for use in the prevention, treatment or therapy of allergic contact dermatitis.

Another aspect of the present disclosure relates to the hydrogel or the topical composition for use in the prevention, treatment or therapy of irritant contact dermatitis.

Another aspect of the present disclosure relates to the hydrogel or the topical composition for use for use in the prevention, treatment or therapy of atopic dermatitis, eczema or seborrheic dermatitis.

Another aspect of the present disclosure relates to the use of the hydrogel or the topical composition as a skin care agent.

Another aspect of the present disclosure relates to the use of the hydrogel or the topical composition as a preventer and/or retardant skin ageing.

In an embodiment, the composition can also be a solution, emulsion, or solid, and the composition can be formulated for topical administration as a lotion, cream, oil, a stick- or bar-shaped solid, a spray, an ointment, a paste, a mousse, a body wash, or a cosmetic.

Another aspect of the present disclosure relates to the use of the hydrogel enriched with the selected molecules for improving the appearance of aging skin or to treat other dermatological conditions associated with inflammation, including atopic dermatitis, eczema, seborrheic dermatitis, irritant contact dermatitis.

### BRIEF DESCRIPTION OF THE DRAWINGS

The following figures provide preferred embodiments for illustrating the disclosure and should not be seen as limiting the scope of invention.

FIG. 1—Effect of the active molecules in the reactivity of the skin allergen DNFB towards the amino acids lysine (LYS) and cysteine (CYS). (A) Glutathione depletion; (B) Lysine depletion.

FIG. 2—Effect of the active molecules on the maturation profile of THP-1 cells evoked by the allergens DNFB and MI assessed by the up-regulation of the co-stimulatory molecules CD54 and CD86. (A) NAC; (B) LYS.

FIG. 3—Effect of the active molecules on the maturation profile of THP-1 cells evoked by the allergens DNFB and MI assessed by the up-regulation of the co-stimulatory molecules CD54 and CD86. (C) CYS; (D) GSH (glutathione); (E) Keratin; (F) Collagen.

FIG. 4—Effect of the active molecules on the maturation profile of THP-1 cells evoked by the allergens DNFB and MI assessed by the up-regulation of the co-stimulatory molecules CD54 and CD86. (G) Methionine; (H) Homocysteine; (I) DMSO; (J)BSA.

FIG. 5—Effect of the active molecule mixtures on the maturation profile of THP-1 cells evoked by DNFB and MI allergens and assessed by the up-regulation of the co-stimulatory molecules CD86 and CD54. (A) NAC+LYS; (B) CYS+LYS.

FIG. 6—Effect of pre-incubation of the active molecules and mixture on the expression of NRF2 dependent genes evoked by DNFB (6h) in human keratinocytes and assessed by real time PCR (Polymerase chain reaction). (A): effect of NAC on HMOX1 (Heme Oxygenase 1), NQ01 ((NAD(P)H Quinone Dehydrogenase 1)) and TRX1 (Thioredoxin-1) expression; (B): effect of LYS on HMOX1, NQ01 and TRX1 expression; (C) effect of NAC+LYS on HMOX1, NQ01 and TRX1 expression.

FIG. 7—Effect of pre-incubation of the active molecules and mixture on the expression of NRF2 dependent genes evoked by DNFB (6h) in human keratinocytes and assessed by real time PCR (Polymerase chain reaction). (D) effect of GSH on HMOX1 and SOD1 (superoxide dismutase 1) expression; (E) effect of HOMO on HMOX1 and SOD1 expression; (F) effect of CYST on HMOX1 and TRX1 expression.

FIG. 8—Effect of the active molecules and of the mixture NAC+LYS on the expression of NRF2 dependent genes evoked by MI (6h) in human keratinocytes and assessed by real time PCR. (A) effect of NAC on HHMOX1, GST, SOD1 and TRX1 expression; (B) effect of LYS on SOD1 and TRX1 expression.

FIG. 9—Effect of the active molecules and of the mixture NAC+LYS on the expression of NRF2 dependent genes evoked by MI (6h) in human keratinocytes and assessed by real time PCR. (C) effect of GSH on HMOX1, NQ01, GST, TRX1 and SOD1 expression; (D) effect of HOMO on GST (glutathione S-transferase), SOD1 and TRX1 expression; (E) effect of CYS on TRX1 expression.

FIG. 10—Effect of the active molecules and of the mixture NAC+LYS on the expression of NRF2 dependent genes evoked by MI (6h) in human keratinocytes and assessed by real time PCR. (F) effect of MET on HMOX1, NQ01, GST, TRX1 and SOD1 expression; (G) effect of NAC+LYS on HMOX1, GST, SOD1 and TRX1 expression.

FIG. 11—Hydrogel formulation and its effect on several parameters: (A) Effect of hydrogel formulation on the maturation profile of THP-1 cells evoked by DNFB and MI allergens and assessed by the up-regulation of the co-stimulatory molecules CD86 and CD54; (B) HPLC quantification of active molecules NAC and LYS, (C) In vitro release testing (IVRT) profile of NAC and LYS in solution and in the hydrogel

FIG. 12—Safety of the hydrogel concerning Human Skin irritation: cytotoxicity in human keratinocytes and irritation potential (A) Irritation test in a reconstructed human tissue model (RHE) (EPISKIN); (B) Representative imagens of the tissues colors after MTT and isopropanol incubations; (C) Metabolic activity of human keratinocytes treated with NAC, LYS, NAC+LYS and hydrogel, accessed by Resazurin Assay—

FIG. 13—In vivo validation of the efficacy of the hydrogel enriched with the best mixture of molecules in avoiding the development of skin sensitization using the murine local lymph node assay (BRDU-LLNA). (A) BRDU-LLNA assay with SI values achieved in the animals treated with DNFB, MI and PPD; (B) Representative histological images of animals treated with DNFB and [100 mM]+DNFB; (C) representative macroscopic imagens of mice ears of control, DNFB, Gel [10 mM]+DNFB, MI and Gel [10 mM]+MI treated animals at the end of the protocol.

### DETAILED DESCRIPTION

The present disclosure relates to a hydrogel for use in the prevention, treatment or therapy of skin allergies comprising an allergen scavenger, wherein said allergen scavenger is a mixture of two amino acid selected from n-acetyl cysteine, lysine, cysteine or their pharmaceutical acceptable salts, or a mixture of two peptide comprising a terminal amino acid selected from n-acetyl cysteine, lysine, cysteine or their pharmaceutical acceptable salts, or mixtures thereof. Furthermore, the present disclosure also relates to topical compositions comprising such hydrogel, and the use in several skin allergies or, namely in the prevention, treatment or therapy of contact dermatitis.

In an embodiment, the present disclosure relates to a hydrogel enriched in several molecules with the capacity of reacting with skin allergens thus rendering them unable to trigger the development of ACD. The development of such hydrogel comprised the following steps:
in vitro screening and in chemico a panel of molecules for their capacity to avoid the adverse outcome pathway evoked by standard human skin allergens, using the non-animal approaches for skin sensitization hazard already approved by the Organization for Economic Co-operation and Development (OECD), specifically the in chemico reactivity towards cysteine and lysine, the maturation of THP-1 cells assessed by the up-regulation of the co-stimulatory molecules CD86 (cluster of differentiation 86) and CD54 (cluster of differentiation 54) and the expression of NRF2 dependent genes in keratinocytes.
Formulation of a hydrogel with the best mixture of molecules disclosed in the previous task;
To discard the potential of the hydrogel enriched with the best mixture of molecules to induce skin irritation using the SkinEthic™ Reconstructed Human Epidermis (RHE) model, in compliance with the OECD Test Guideline No. 439.
in vivo validation of the efficacy of the hydrogel enriched with the best mixture of molecules in avoiding the development of skin sensitization using the murine local lymph node assay (LLNA);

In an aspect, the invention features a hydrogel or a topical composition for use the prevention, treatment or therapy of a skin allergy or as a skin care agent by administering, to a patient, an effective amount of a hydrogel or a topical composition comprising any of the compositions herein described. The patient may be suffering from (i.e., may have been identified or diagnosed as having): atopic dermatitis, eczema, disseminated superficial actinic porokeratosis, seborrheic dermatitis, allergic contact dermatitis, irritant contact dermatitis, lichen planus, or may have inflamed skin due to a rash, hives, a bum, haemorrhoids, contact with poison ivy or poison oak, an insect sting, a cut, or vitiligo.

In other embodiments, the patient may have aging skin, dry skin, itchy skin, red skin, or cracked skin. In other embodiments, the patient can have acquired or congenital icthvosis or may have Netherton's syndrome, epidermolytic ichthyosis, lamellar ichthyosis, Mai de Meieda, harlequin ichthyosis, or erythrokeratodermia variablis.

In another aspect, the topical compositions of the present invention can include, in addition to the aforementioned components, a wide variety of additional oil-soluble materials and/or water-soluble materials conventionally used in topical compositions, at their art-established levels. Water-soluble materials include humectants, proteins and polypeptides, preservatives, and alkaline agents. In addition, any of the present topical compositions can contain conventional cosmetic adjuvants, such as dyes, opacifiers (e.g., titanium dioxide), pigments and perfumes (however, as noted, in some embodiments, compositions can be free of odor-masking materials).

Within the present disclosure, a peptide is any compound produced by amide formation between a carboxyl group of one amino acid and an amino group of another. The amide bonds in peptides may be called peptide bonds. The word peptide usually applies to compounds whose amide bonds are formed between C-1 of one amino acid and N-2 of another (sometimes called eupeptide bonds), but it includes compounds with residues linked by other amide bonds (sometimes called isopeptide bonds). Peptides with fewer than about 10-20 residues may also be called oligopeptides; those with more, polypeptides. Polypeptides of specific sequence of more than about 50 residues are usually known as proteins, but authors differ greatly on where they start using this term.

An amino-acid residue is when two or more amino acids combine to form a peptide, the elements of water are removed, and what remains of each amino acid is called an amino-acid residue.

### Materials and Methods

### In Chemico Reactivity Towards Lysine and Cysteine:

In an embodiment, a Lysine depletion Protocol—“in house development protocol” was applied, comprising: Dissolve 0.01 g of Poly-L-Lysine Hydrobromide (PLL) (Sigma, P1399) in 100 mL of purified water (use within 2 months). Filter the solution with a syringe filter to remove aggregates and store ate 4° C. for further use. For plate coating add 50 μL of PLL to each well of the 96 black bottom glass microplate (Corning ref 45806) and incubate for 2 hours at 37° C. or 24 h at room temperature.

In an embodiment, after the coating procedure, the contents of the plate were discarded into a sink and the plate is washed 3 times with 200 μL of PBS per well. Blot any residual PBS by firmly tapping the plate upside-down on absorbent paper. 20 mM of each potential ACD inhibitory molecule/mixture is added and incubated with 70 μl of the strong skin sensitizer 1-fluoro-2,4-dinitrobenzene (DNFB) (5 mM in DMSO) for 1h; the mixture was transferred to the plate and incubated for 24 h at 37° C. The contents of the plate were discarded and the wells were washed 3 times with PBS as previously reported. 70 μL of 10 μM Fluorescein-5-Ex-Succinimidyl Ester (FSE) were added and incubated for 1 h in the dark at 37° C. The contents of the plate were discarded; the wells were washed 3 times with PBS as previously stated. The fluorescence was read in a microplate reader (Exc 485±20 nm and Em 530±30 nm). Synergy™ HT plate reader (BioTek Instruments, Inc., Winooski, VT, USA): Bottom read with a sensibility of 50.

### Control and Test Chemical Preparation:

In an embodiment, DNFB is used as the positive control for the assay in every assay run. For the negative control, LA (lactic acid) is used and, for the reference control, three types of reference controls are included in each study.

In an embodiment, Reference control A is made with the solvent DMSO, with incubation with FSE and is used to calculate the maximum fluorescence or absorbance in the presence of LYS. The percentage of peptide depletion in each sample must be normalized to this control.

In an embodiment, Reference control B is made with the solvent DMSO, without incubation with FSE and is used to calculate the assay blank. Reference control C is made with the test chemicals, without incubation with FSE and is used to verify the test chemical impact on the percentage of peptide depletion.

### Test Chemicals

In an embodiment, Test chemicals were pre-weighted into 1.5 mL test tubes and dissolved in 1 mL of DMSO to prepare a 100 mM solution immediately before use. The weight of the test chemical to be added to the vial was determined based on the molecular weight and purity. The final concentration of the test chemical in each assay is 5 mM.

The assay should include the following conditions:

| | | |
|---|---|---|
| A | Reference Control A | 70 μL DMSO + 70 μL FSE |
| B | Reference Control B | 70 μL DMSO + 70 μL PBS |
| C | Reference Control C | 70 μL test chemical + 70 μL PBS |
| D | Positive Control, PC | 70 μL DNFB + 70 μL FSE |
| E | Negative Control, NC | 70 μL LA + 70 μL FSE |
| F | Assay with Test chemical | 70 μL test chemical + 70 μL FSE |

In an embodiment, for the data Analysis the percentage of peptide depletion is determined by using the following formula:

$$\% Peptide Depletion = [ 1 - ( \frac{F - C}{A - B} ) \times 100$$

### Cysteine Depletion Protocol

In an embodiment, Cysteine depletion Protocol-Glutathione in solution depletion protocol—“in house development protocol” was applied:
In a test tube, mix 10 μL of the 100 mM test chemical solution with 190 μL of GSH solution (20 μL of GSH 2 mM and 170 μL of PB) and Incubated at RT, protected from light, for 1 h in a tube roller (40 rpm)
   Vf=200 L; [test chemical]f=5 mM; [GSH]f=0.2 mM;
   20 μL of Ellman's reagent 10 mM, a thiol sensitive colorimetric reagent was added. Mix gently by tapping manually in the test tube and incubate for 10 min at RT, in a test tube rotator protected from light;
   The absorbance was read in a microplate reader at 405 nm.
   Note: For each assay, a fresh Glutathione stock of 50 mM (0.00768 g in 500 μL of PBS) and the second stock of 2 mM in PBS (2 μL of the 0.5 M solution+48 μl of PBS) were freshly prepared.

### Control and Test Chemical Preparation

In an embodiment, Control and Test Chemical Preparation was applied:
DNFB is used as the positive control for the assay in every assay run. For the negative control LA is used and for the reference control three types of reference controls are included in each study:
Reference control A is made with the solvent DMSO, with incubation with Ellman's reagent and is used to calculate the maximum fluorescence or absorbance in the presence of CYS containing peptides. The percentage peptide depletion in each sample must be normalized to this control.
Reference control B is made with the solvent DMSO, without incubation with Ellman's reagent and is used to calculate the assay blank.
Reference control C is made with the test chemicals, without incubation with Ellman's reagent and is used to verify the test chemical impact on the Percent Peptide Depletion.

### Test Chemicals

Test Chemicals: In an embodiment, test chemicals were pre-weighted into 1.5 mL test tubes and dissolved in 1 mL of DMSO to prepare a 100 mM solution immediately before use. The weight of the test chemical to be added to the vial is determined based on the molecular weight and purity. The final concentration of the test chemical in each assay is 5 mM. The assay should include the following conditions:

| | | |
|---|---|---|
| A | Reference Control A | 20 μL of GSH + 170 μL PB |
| | | 10 μL DMSO |
| | | and further colorimetric reagent |
| B | Reference Control B | 20 μL of GSH + 170 μL PB |
| | | 10 μL DMSO |
| C | Reference Control C | 20 μL of GSH + 170 μL PB |
| | | 10 μL test chemical |
| D | Positive Control, PC | 20 μL of GSH + 170 μL PB + |
| | | 10 μL DNFB |
| | | and further colorimetric reagent |
| E | Negative Control, NC | 20 μL of GSH + 170 μL PB + |
| | | 10 μL LA |
| | | and further colorimetric reagent |
| F | Assay with Test Chemical | 20 μL of GSH + 170 μL PB + |
| | | 10 μL test chemical |
| | | and further colorimetric reagent |

In an embodiment, for the data analysis, the percent depletion of peptide is determined by using the following formula:

$$\% Peptide Depletion = [ 1 - ( \frac{F - C}{A - B} ) \times 100$$

### Maturation of THP-1 Cells Assessed by the Up-Regulation of the Co-Stimulatory Molecules CD86 and CD54

In an embodiment, the THP-1 cell line has been used as a dendritic cell (DC) surrogate for skin sensitizers hazard, following the OECD guidelines [35]. The human monocytes (THP-1, ATCC TIB-202, Manassas, VA, USA) cell line is cultured and maintained at a cell density between 2×10⁵ and 1×10⁶ cells/mL in RPMI 1640 supplemented with 10% (v/v) inactivated FBS, 1% (v/v) antibiotic solution, 1.5 g/L sodium bicarbonate, 25 mM glucose, 10 mM HEPES ((4-(2-hydroxyethyl)-1-piperazineethanesulfonic acid)) and 1 mM sodium pyruvate. Cells are cultured in 75 cm² flasks and maintained in a humidified 5% CO₂-95% air atmosphere at 37° C., and the medium was changed every 2-3 days. The THP-1 cells are stabilized overnight at a density of 5×10⁵ cells/mL. On the day after, cells are seeded in 12-well plates at a density of 8×10⁵ cells/well and incubated for 24 h with medium (control), Dimethyl sulfoxide (DMSO) (allergens solvent) or with 20 mM of each inhibitor of ACD (NAC), (LYS) or mixture (NAC+LYS). In addition, sensitization is induced with the strong skin allergen 1-fluoro-2,4-dinitrobenzene (DNFB) (8 μM) or Methylisothiazolinone (MI) (100 μM) for 24 h. At the end of the incubation period, cells are washed with ice-cold PBS supplemented with 1% (v/v) inactivated FBS, centrifuged at 300×g for 5 min at 4° C. and pellets are dissolved in ice-cold PBS supplemented with 1% (v/v) inactivated FBS. Then, 100 L of the cell suspension are incubated with, or without, 3 μL of anti-human CD54 antibody (#353111, Biolegend, San Diego, CA, USA) or anti-human CD86 (#305414, Biolegend, San Diego, CA, USA) antibody for 30 min at 4° C. After that, cells are washed and resuspended again in ice-cold PBS supplemented with 1% (v/v) inactivated FBS and analyzed using a flow cytometer (BD Accuri™ C6 Flow Cytometer, BD Biosciences, San Jose, CA, USA). A total of 1×10⁴ living cells are considered for analysis. The CD86 and CD54 levels are analyzed by flow cytometry with the acquisition channels FL1 and FL4. Based on the geometric mean fluorescence intensity (MFI), the relative fluorescence intensity (RFI) of CD86 and CD54 for positive control cells and chemical-treated cells is calculated according to the following equation:

$$RFI = \frac{\begin{matrix}MFI of chemical treated cells - \\ MFI of chemical treated unstained cells\end{matrix}}{MFI of control treated cells - MFI of control unstained cells} \times 100$$

In an embodiment, results from at least three independent experiments are expressed as a percentage (%) of RFI obtained in control, which was considered 100%.

### Expression of NRF2 Dependent Genes in Keratinocytes

In an embodiment, following the OECD guidelines for skin sensitization assessment, the increased expression of Nrf2-dependent genes in the human keratinocytes cell line (HaCaT, CLS 300493, Eppelheim, Germany) has been used for skin sensitizers hazard [36]. HaCaT is cultured with Dulbecco's Modified Eagle's Medium (DMEM) (Sigma-Aldrich, St. Louis, MO, USA), supplemented with 10% (v/v) heat-inactivated fetal bovine serum (FBS), 1% (v/v) antibiotic solution (100 U/mL penicillin, 100 μg/mL streptomycin) (Gibco, Carlsbad, CA, USA), 3.7 g/L sodium bicarbonate and 1 mM sodium pyruvate (Sigma-Aldrich, St. Louis, MO, USA).

In an embodiment, HaCaT cells are seeded in 6-well plates at a density of 6×10⁵ cells/well and allowed to adhere for 24 h. After that, cells are incubated for 24 h with exposure medium (control) or treated with 10 mM of NAC, 10 mM of LYS, 8 μM of DNFB, 100 μM of MI, or 10 mM+DNFB, 10 mM LYS+DNFB and 10 mM+MI, 10 mM LYS+MI for 6 h or 24 h.

In an embodiment, total RNA (ribonucleic acid) is extracted from HaCaT treated cells, as previously described by our group [37], with NZYol reagent, according to the manufacturer's instructions and its concentration is determined by OD260 measurement using a NanoDrop spectrophotometer (Thermo Scientific, Wilmington, DE, USA). Samples are stored in RNA Storage Solution at −80° C. until use. Total RNA (1 g) is reverse-transcribed using the iScript Select cDNA Synthesis Kit, and real-time reverse transcriptase-polymerase chain reaction (RT-PCR) reactions are performed, in duplicate for each sample, on a Bio-Rad MyCycler iQ5 (Hercules, CA, USA), as previously described by our group [38].

In an embodiment, after amplification, a threshold is set for each gene and Ct values are calculated for all samples. Gene expression changes are analyzed using Bio-Rad CFX Maestro 1.1 system software (Hercules, CA, USA). The results are normalized using Hprt-1 as the reference gene. Primer sequences are designed using Beacon Designer software version 7.7 (Premier Biosoft International, Palo Alto, CA, USA) and thoroughly tested.

### List of Used Primers:

| | | | | Qualifier | |
|---|---|---|---|---|---|
| | Organism | | Molecule | Molecule | |
| Seq. ID | name | Sequence Name | type | Type | Residues |
| Seq. ID. 1 | Artificial | HPRT1 | DNA | Other DNA | 5′ |
| | sequence | (hypoxanthine phos- | | (Forward | tgacactggcaaa |
| | | phoribosyltransferase | | primer) | acaatg 3′ |
| | | 1)-Forward | | | |
| | | primer | | | |
| Seq. ID. 2 | Artificial | HPRT1 | DNA | Other DNA | 5′ |
| | sequence | (hypoxanthine phos- | | (Reverse | ggcttatatccaac |
| | | phoribosyltransferase | | primer) | acttcg 3′ |
| | | 1)-Reverse primer | | | |
| Seq. ID. 3 | Artificial | HMOX1 (heme | DNA | Other DNA | 5′cctgagtttcaa |
| | sequence | oxigenase 1)-Forward | | (Forward | gtatcc 3′ |
| | | primer | | primer) | |
| Seq. ID. 4 | Artificial | HMOX1 (heme | DNA | Other DNA | 5′aacaacagaac |
| | sequence | oxigenase 1)-Reverse | | (Reverse | acaacaa 3′ |
| | | primer | | primer) | |
| Seq. ID. 5 | Artificial | NQO1 | DNA | Other DNA | 5′gagtctgttctg |
| | sequence | (quinone | | (Forward | gcttat 3′ |
| | | oxidoreductase 1)- | | primer) | |
| | | Forward primer | | | |
| Seq. ID. 6 | Artificial | NQO1 | DNA | Other DNA | 5′ |
| | sequence | (quinone | | (Reverse | aactggaatatca |
| | | oxidoreductase 1- | | primer) | caaggt 3′ |
| | | Reverse primer | | | |
| Seq. ID. 7 | Artificial | TRX1 | DNA | Other DNA | 5′gccgctcgtcag |
| | sequence | (Thioredoxin-1)- | | (Forward | actccag 3′ |
| | | Forward primer | | primer) | |
| Seq. ID. 8 | Artificial | TRX1 | DNA | Other DNA | 5′gcagcgtccaa |
| | sequence | (Thioredoxin-1)- | | (Reverse | ggcttcc 3′ |
| | | Reverse primer | | primer) | |
| Seq. ID. 9 | Artificial | SOD1 | DNA | Other DNA | 5′ |
| | sequence | (superoxide dismutase | | (Forward | tggcttgtggtgta |
| | | 1)-Forward primer | | primer) | attg 3′ |
| Seq. ID. 10 | Artificial | SOD1 | DNA | Other DNA | 5′tagcaggataa |
| | sequence | (superoxide dismutase | | (Reverse | cagatgagt 3′ |
| | | 1)-Reverse primer | | primer) | |
| Seq. ID. 11 | Artificial | GST | DNA | Other DNA | 5′ |
| | sequence | (glutathione S- | | (Forward | ggaggcaagacct |
| | | transferase P)- | | primer) | tcatt 3′ |
| | | Forward primer | | | |
| Seq. ID. 12 | Artificial | GST | DNA | Other DNA | 5′ |
| | sequence | (glutathione S- | | (Reverse | atggatcagcagc |
| | | transferase P)- | | primer) | aagtc 3′ |
| | | Reverse primer | | | |

In an embodiment, since RT-PCR results are presented as ratios of chemical-treated samples over untreated (control) cells, a two-base logarithmic transformation is used to make observations symmetric and closer to a normal distribution. If x represents the fold change of a gene in one sample, then the two-base logarithmic transformation (log₂(x)) is ln(x)/ln(2). Therefore, fold changes of 2 and 0.5 correspond to mean log 2 values of 1 and −1, respectively.

### Formulation of the Hydrogel

In an embodiment, a hydrogel formulation was prepared following a conventional manufacturing method. Briefly, and considering a quality by design approach, 20 and 100 mM of NAC and LYS, respectively, are dissolved in the initial aqueous solution, containing Disodium edetate (Na₂EDTA) (0.01-0.1% w/w) and benzoic acid (0.1% w/w), to which, after 30 min of mechanical agitation, Carbopol® Ultrez NF 10 (Carbomer Interpolymer Type A) (0.5-2% w/w) is added (HS-30D mechanical stirrer, Witeg, Germany), at 800 rpm during 1 h to enable complete polymer hydration. 30 ml of isopropanol and glycerol (0-10 ml) are then incorporated into the formulation and the volume is finally adjusted with water to 100 mg. NaOH 4M solution is used to adjust the final pH of the formulation to 5.5 (digital pH C3010 Multiparameter Analyzer (Consort bvba, Belgium)). Each hydrogel was freshly prepared before each experiment and stored at 25° C. The following table depicts the composition of the hydrogel formulation.

| | Concentration |
|---|---|
| Component | (% w/w) |
| NAC | 0.2-2% |
| LYS | 0.2-2% |
| Na₂EDTA | 0.01-0.1% |
| Benzoic Acid | 0.1% |
| Carbopol ® Ultrez NF 10 | 0.5-2% |
| Isopropanol | 30% |
| Glycerol | 0-10% |
| Water | Up to 70% |

### In Vitro Release Testing (IVRT)

In an embodiment, release studies of the hydrogels were performed on static vertical Franz cells (PermeGear, Inc., USA), with a diffusion circular area of 0.636 cm² and a receptor compartment of 5 mL. For that, infinite dose conditions (1 g) of the formulations were applied in the upper compartment, separated from the receptor by a cellulose membrane (MWCO≈14 000 Da, flat width 33 mm, Sigma-Aldrich), previously hydrated in ultrapurified water for at least 30 min. Before the experiments, the system was equilibrated for a minimum of 30 min. The release medium was composed of phosphate buffer pH 5.5 (guaranteeing the maintenance of sink conditions) and continuously stirred at 600 rpm, at 37° C. A circulating water bath maintained the membrane surface temperature at 32° C. During the release studies, the donor compartment, as well as the receptor sampling arm, were carefully covered to avoid medium evaporation, as well as to conduct the experiments under occlusive conditions. At predetermined time points (0.25, 0.5, 0.75, 1, 2, 3, 4 and 6 h), 300 μl of the medium were withdrawn and immediately replenished with fresh medium. NAC and LYS were quantified by HPLC (High-performance liquid chromatography). Release studies were conducted considering n=6 samples.

In an embodiment, the cumulative amount of NAC and LYS released over time was calculated according to the equation:

$$Qn ( µg / {cm}^{2} , ) = \frac{Cn \times V 0 + {\sum }_{i = 1}^{n = 1} Ci \times Vi}{A}$$

where Cn is the API (active pharmaceutical ingredient) concentration of the receptor medium at each sampling time, Ci is the API concentration of the ith sample, A the effective diffusion area (0.636 cm²), and VO (5 mL) and Vi (300 μL) the volumes of the receptor compartment and the collected sample, respectively.

In an embodiment, the release rates were retrieved from the slope of the linear regression obtained by plotting the cumulative amount of API diffused per cm² as a function of the square root of time. Additionally, mass balance studies were performed to characterize the extent of dose depletion, according to the equation:

$$D D ( \% ) = \frac{Q f}{Q i} \times 100$$

where Qf reflects the average cumulative amount released at the end of the assay and Qi the initial amount of API placed on the membrane.

### In Vitro Permeation Studies (IVPT)

In an embodiment, the same vertical diffusional system of IVRT studies was used to evaluate the potential in vitro penetration of the active molecules. Human surgical skin pieces (female, type 3 skin, 57 years old, arm tissue) were surgically removed at Central Lisbon University Hospital Centre, after the approval of the Bioethics Committee, and processed as described in (Miranda et al., 2020). Written informed consent was obtained from the participant (Process number 447/2017). One day before the experiments, frozen epidermal specimens were left to thaw at room temperate. Afterwards, the skin sheets, with the stratum corneum side facing up, were gently placed on glass flasks filled with ultrapurified water and left to stabilize overnight, at 4° C. On the day of the experiments, the epidermis was carefully transferred to each Franz cell, with barrier integrity being tested by assessing the transepidermal water loss (TEWL), recurring to a vapometer (Delfin Technology, Kuopio, Finland). Indications of stretch marks, physical damage, or a TEWL value higher than 20.0 g/m2/h led to the rejection of the epidermic piece (Shin et al., 2020). Similarly, to IVRT, infinite dose conditions (1 g) of the formulations were applied to the donor compartment. The receptor medium, composed of 5 ml of phosphate buffer pH 5.5, was continuously stirred at 600 rpm while all experiments were conducted in a temperature-controlled water bath to ensure a skin surface of 32±1° C. Nonetheless, the assay was conducted under non-occlusive conditions, to mimic clinical use. At predetermined time points (0.25, 0.5, 0.75, 1, 2, 3, 4, 6, 8 and 24 h), 300 μL of the medium were withdrawn and immediately replenished with fresh medium. NAC and LYS were quantified by RP-HPLC, as described in the section above. Release studies were conducted considering five replicates.

### NAC and LYS Quantification

In an embodiment, NAC and LYS were quantified by reversed-phase high-pressure liquid chromatography (RP-HPLC), using a Shimadzu LC-2010HT chromatographic system, equipped with an LC-20AD quaternary pump, a SIL-20AHT autosampler, a CTO-10AS over and an SPD-M2OA photodiode array detector. The separation of the analytes was achieved on a Surf C8 column, with 3 μm of particle size, 4.6 mm of internal diameter and 150 mm of length (ImChem Sarl, Portugal) under the following conditions: isocratic mode, at 30° C., with a mobile phase composed by 95:5 (v/v) phosphate buffer 25 mM pH 3.0: acetonitrile, at 0.8 mL/min. Using an injection volume of 50 μL, NAC and LYS were determined at 200 and 215 nm and eluted at 5.4 and 2.2 min, respectively.

### Skin Irritation

In an embodiment, skin irritation is evaluated using the SkinEthic™ Reconstructed Human Epidermis (RHE) model (EPISKIN Laboratories, Lyon, France), in compliance with the OECD Test Guideline No. 439-In Vitro Skin Irritation: Reconstructed

Human Epidermis Test Method [39]. The RHE model consists of a differentiated three-dimensional epi-dermal tissue of human keratinocytes grown on a 0.5 cm² surface inert polycarbonate filter in a chemically defined medium at the air-liquid interface. Two specific media provided by EPISKIN are used: maintenance and growth media. On the day of receipt, the SkinEthic™ RHE inserts are transferred into 6-wells plates filled with 1 ml of maintenance medium and stored in an incubator at 37° C., under a 5% CO₂-95% air atmosphere, for 24 h. For the experiments, SkinEthic™ RHE inserts are placed in a maintenance medium in 24-well plates (300 μL/well) and then the hydrogels, with or without 10 mM NAC and 10 mM LYS, are topically applied on the surface of an insert on three tissues replicates, for 42 min at RT. The experiment also includes other inserts treated with PBS or 5% (w/v in water) SDS used as negative and positive controls, respectively. After the exposure time, the SkinEthic™ RHE inserts are rinsed 25 times with 1 mL PBS and transferred to 6-well plates with growth medium (2 mL/well) to be incubated for 42 h at 37° C., under a 5% CO₂ with 95% air atmosphere. Tissue viability is assessed using the MTT (3-(4,5-dimethylthiazol-2-yl)-2,5-diphenyl-2H-tetrazolium bromide) assay. For that, tissues are incubated with MTT (1 mg/mL) at 37° C. for 3h; then the MTT solution was removed, and formazan crystals were dissolved with isopropanol. The absorbance was measured at 570 nm using a SpectraMax Plus 384 Spectrophotometer. The results achieved on three tissue replicates are expressed as a percentage (%) of the absorbance value obtained in the negative control, which is considered 100%, and were graphically presented as % of tissue viability. If the tissue viability value is <50%, the samples are classified as irritants.

In vivo validation of the efficacy of the hydrogel enriched with the best mixture of molecules, to avoid the development of skin sensitization using the murine local lymph node assay (LLNA)

In an embodiment, the fourth key event in the adverse outcome pathway (AOP) for skin sensitization is T cells priming, proliferation and activation at the lymph nodes. This event can be confirmed using the in vivo test Local lymph node assay (LLNA), which is evaluated by flow cytometry (FCM) using the OECD Guideline for the testing of chemicals: BRDU (bromodeoxyuridine) ELISA (enzyme linked immunosorbent assay) or FCM 442B, as previously described and revised on OECD TG 442B [41].

In an embodiment, a total of 52 female BALB/c mice with 8-12 weeks, where weight variation of the animals is minimal and does not exceed 20% of the mean weight, are purchased from Charles River Laboratories, France. The animals are housed at a temperature of 22° C. and relative humidity of 30-70%. The room is lit with artificial light for 12 h/day. The animals are free to access solid diets and sterilized drinking water. During the entire procedure, the daily weight record is made to assess the welfare of the animals. After the five days acclimatization period, the animals are randomly divided into 14 experimental groups:
Group only injected with BrdU (negative, negative control) (group 1) (n=6)
Group topically applied with 0.2 ml of hydrogel vehicle (10 mM): hydrogel without the molecules (group 2), followed by application of 25 μl 0.25% DNFB (n=4)
Group topically applied with 0.2 ml of hydrogel vehicle (100 mM): hydrogel without the molecules (group 3), followed by application of 25 μL 0.25% DNFB (n=6)
Group topically applied with 25 μL of the allergen vehicle control (AOO) acetone: olive oil (4:1, v/v), (group 4) (n=6)
Group topically applied with 25 μL allergen 0.25% DNFB in acetone: olive oil (4:1, v/v) (positive control), (group 5) (n=5)
Group topically applied with 0.2 mL hydrogel enriched with molecules 10 mM followed by application of 25 μL 0.25% DNFB, (group 7) (n=5)
Group topically applied with 0.2 mL hydrogel enriched with molecules 100 mM followed by application of 25 μL 0.25% DNFB, (group 9) (n=5)
Group topically applied with 25 μl allergen 2.5% MI in acetone: olive oil (4:1, v/v) (positive control), (group 10) (n=2)
Group topically applied with 0.2 mL of hydrogel vehicle (10 mM): hydrogel without the molecules (group 11), followed by application of 25 μl 2.5% MI in acetone: olive oil (4:1, v/v), (group 11) (n=3) 1 Group topically applied with 0.2 mL hydrogel enriched with molecules 10 mM followed by application of 25 μL 2.5% MI in acetone: olive oil (4:1, v/v), (group 12) (n=3)
Group topically applied with 0.2 mL of hydrogel vehicle (10 mM): hydrogel without the molecules (group 11), followed by application of 25 μL 0.5% PPD in acetone: olive oil (4:1, v/v), (group 13) (n=1)
Group topically applied with 0.2 mL hydrogel enriched with molecules 10 mM followed by application of 25 μL 0.5% PPD in acetone: olive oil (4:1, v/v), (group 14) (n=1)

In an embodiment, all the tested materials were made by the same method freshly before every administration.

In an embodiment, on days 1, 2, and 3, 25 μL of DNFB/vehicle control and/or 0.2 ml of the hydrogel, are spread on the back of each ear daily.

In an embodiment, on day 5, animals are intraperitoneally injected with 100 μl of BrdU mixture (20 mg/mL) in PBS. On day 6, mice are euthanized, and the auricular-lymph nodes are collected. Lymph nodes are mashed using a cell scraper SPL, Pocheon, Gyeonggi-do, Korea) and cell strainer with 70 μm nylon meshes white frame colour, sterile (Corning), to obtain the lymph node cells (LNCs). LNCs are stained with trypan blue (Sigma) and counted using a hemocytometer. LNCs (1.5×10⁶ cells/mL) are prepared using a commercially FITC BrdU Flow Kit-Catalog No. 559619 (BD Biosciences, San Jose, CA, USA) and further analyzed by flow cytometry using a BD Accuri™ C6 Flow Cytometer (BD Biosciences, San Jose, CA, USA). Stimulation index (SI) values for each sample are calculated using the formula described in the OECD test guideline (TG 442B) [41]. If the SI values are above 2.7 the test substances are classified as sensitizers. SI is calculated according to the following equation:

$$Stimulation index ( SI ) = \frac{\begin{matrix}Number of BrdU - \\ positive LNCs from each mouse exposed to a test substance\end{matrix}}{\begin{matrix}Mean number of BrdU - \\ positive LNCs in the vehicle control group\end{matrix}}$$

Several parameters are also evaluated: body weight, ear thickness (macroscopically), and lymph node HE staining.

### Statistical Analysis

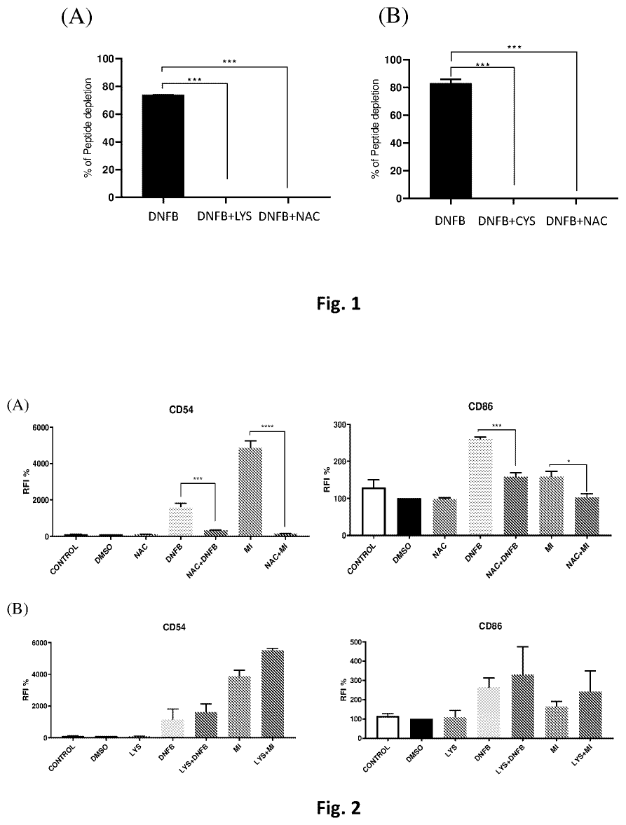

In an embodiment, data were analyzed using the GraphPad Prism 8.0 (Graph-Pad software ver. 8, San Diego, CA, USA). All data are expressed as the mean±standard error of the mean (SEM). In chemico reactivity, dendritic cells maturation and NRF2 activation data is compared by one-way ANOVA compared by post hoc Turkey's pair-wise comparisons. LLNA: BrdU-FCM assay data are compared by unpaired t-test. A value of p<0.05 is considered statistically significant. FIG. 1 shows that n-acetyl cysteine (NAC), lysine (LYS) and cysteine (CYS) significantly inhibit the reactivity of the strong allergen DNFB towards lysine and cysteine, thus preventing the first key event of the development of ACD

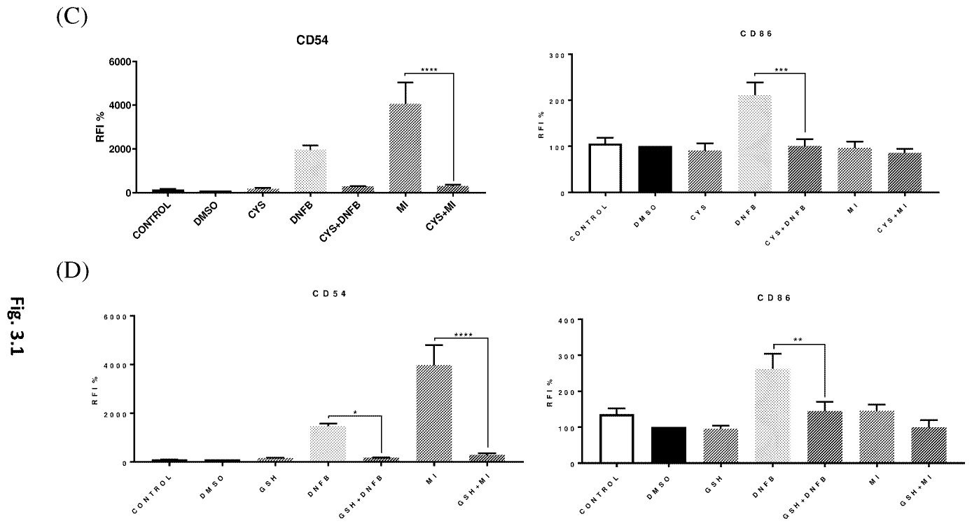

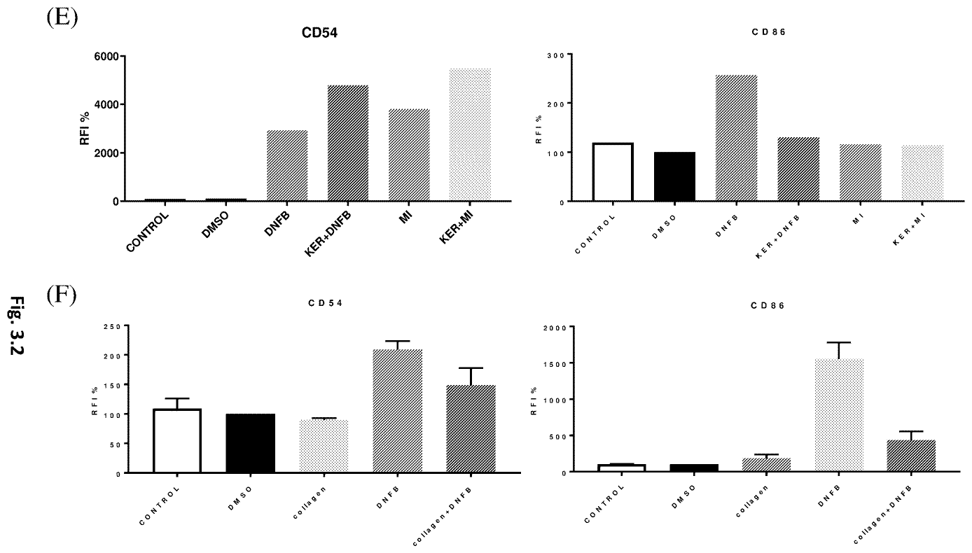

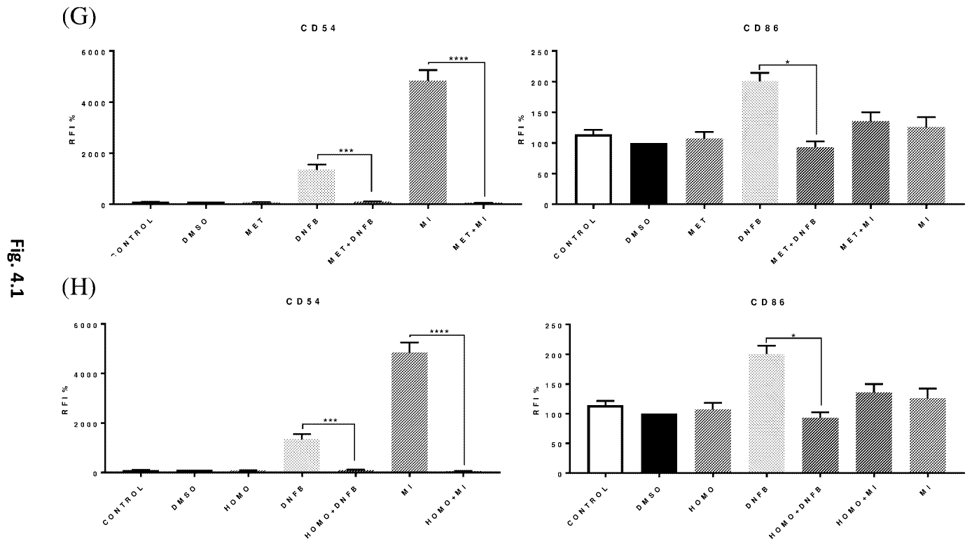

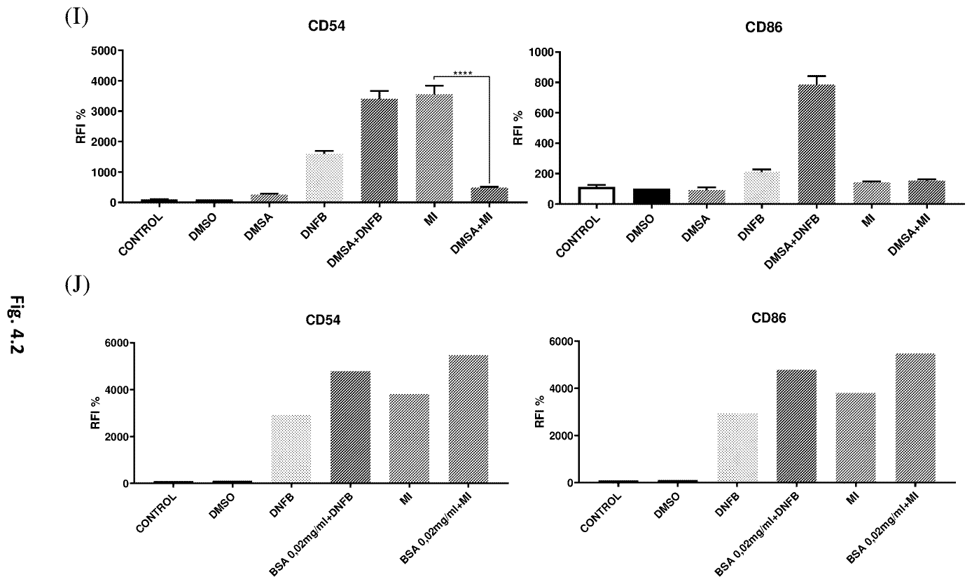

FIG. 2-4 shows the inhibitory effect of NAC, LYS, CYS, GSH, keratin, collagen, methionine (MET), HOMO (homocysteine), DMSA (2,3 dimercaptosuccinic acid) and BSA (bovine serum albumin) on the maturation of THP-1 cells induced by the allergens DNFB and MI, assessed as the intensity fluorescence ratio (RFI) of co-stimulatory molecules CD54 and CD86. The treatment with DNFB and MI, which are strong skin sensitizers, significantly increases the levels of CD54 and CD86. In contrast, NAC, LYS, CYS, GSH, keratin, collagen, methionine, homocysteine, DMSA and BSA when incubated alone do not significantly modify the levels of the co-stimulatory molecules CD54 and CD86, thus discarding the potential of these molecules to trigger skin sensitization. Moreover, the Overall, pre-incubation of each inhibitory molecule before the treatment with 24 h DNFB and MI mitigates cells maturation with significantly decrease CD54 and CD86 maturation levels for NAC, CYS, GSH, MET, HOMO AND DMSA. The results demonstrate that some active molecules prevent the third key event of the development of ACD. The results are expressed as at least five independent experiments. * p<0.05; ** p<0.01 *** p<0.001; **** p<0.0001. N-Acetyl Cysteine (NAC), Lysine (LYS), Cysteine (CYS), Glutathione (GSH), keratin (KER), collagen, methionine (MET), homocysteine (HOMO), 2,3 Dimercaptosuccinic Acid (DMSA), Bovine serum albumin (BSA) and Dimethyl sulfoxide (DMSO).

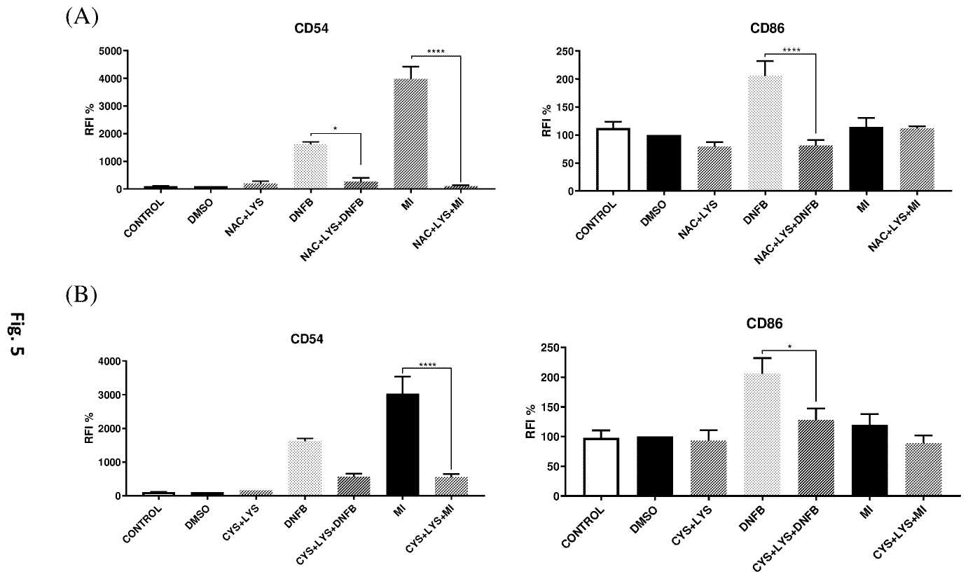

FIG. 5 shows the inhibitory effect of (A) NAC+LYS and (B) CYS+LYS on the maturation of THP-1 cells induced by DNFB and MI allergens. The treatment with 24 h DNFB and MI, which are strong skin sensitizers, increase the levels of CD54 and CD86. In contrast, NAC+LYS and CYS+LYS alone do not significantly modify the levels of the co-stimulatory molecules CD54 and CD86, thus discarding the potential of these molecules mixtures to trigger skin sensitization. Furthermore, pre-incubation with NAC+LYS and CYS+LYS before the treatment with DNDB or MI significantly mitigates cells maturation, with the decrease of CD54 and CD86 maturation levels. The results demonstrate that the mixtures of these active molecules prevent the third key event of the development of ACD. The results are expressed as at least five independent experiments. * p<0.05; ** p<0.01 *** p<0.001; **** p<0.0001.

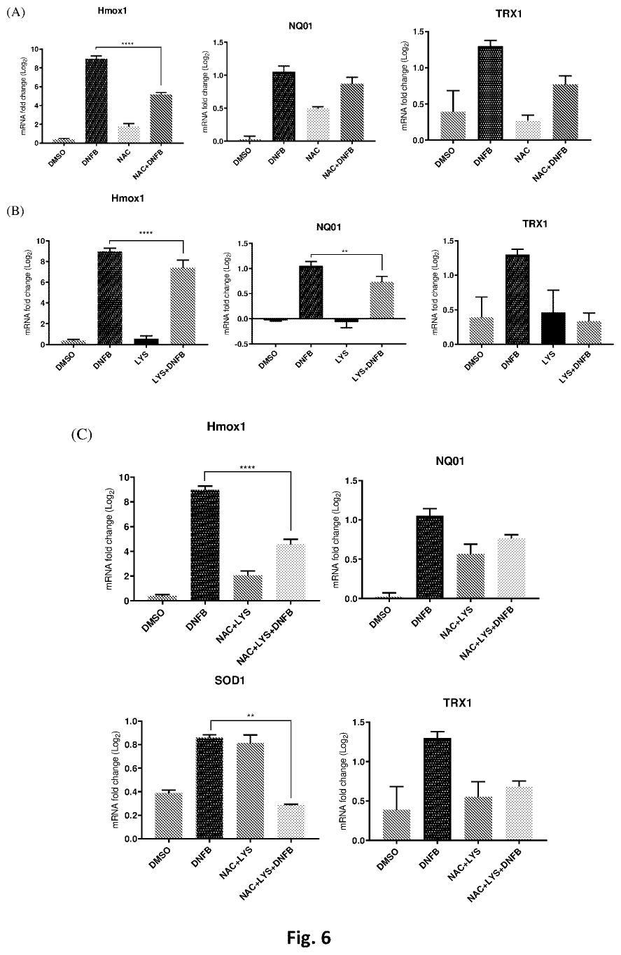

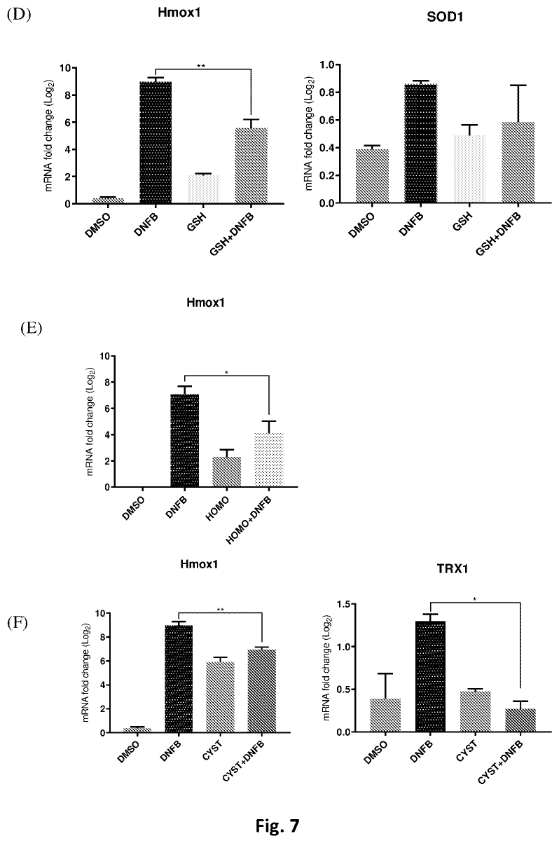

FIG. 6-7 shows the effect of NAC, LYS, NAC+LYS, GSH, HOMO and CYS on the expression of Nrf2-dependent genes in human keratinocytes, namely HMOX1, NQO1, TRX1 and SOD1 expression levels, after 6 h of DNFB incubation. Overall, there is a decrease on gene expression on all analysed genes when the cells that were incubated with the active molecules before the treatment with DNFB, in comparison with cells treated with DNFB alone, particularly with a significantly decrease of HMOX1 when NAC, LYS, NAC+LYS, GSH, HOMO and CYS are incubated before DNFB, NQO1 when, LYS is incubated before DNFB, SOD1 when NAC+LYS, GSH, HOMO are incubated before DNFB and TRX1 when CYS is incubated before DNFB. The results demonstrate that the active molecules prevent the second key event of the development of ACD. The results are expressed as relative fold changes compared to control, solvent of the allergens is DMSO and is also represented in the graph and represents the mean±SEM of at least 3 independent experiments. Statistical analysis was made by one-way analysis of variance (ANOVA) followed by Dunnett's multiple comparison test. * p<0.05; ** p<0.01 *** p<0.001; **** p<0.0001 significantly different compared to control.

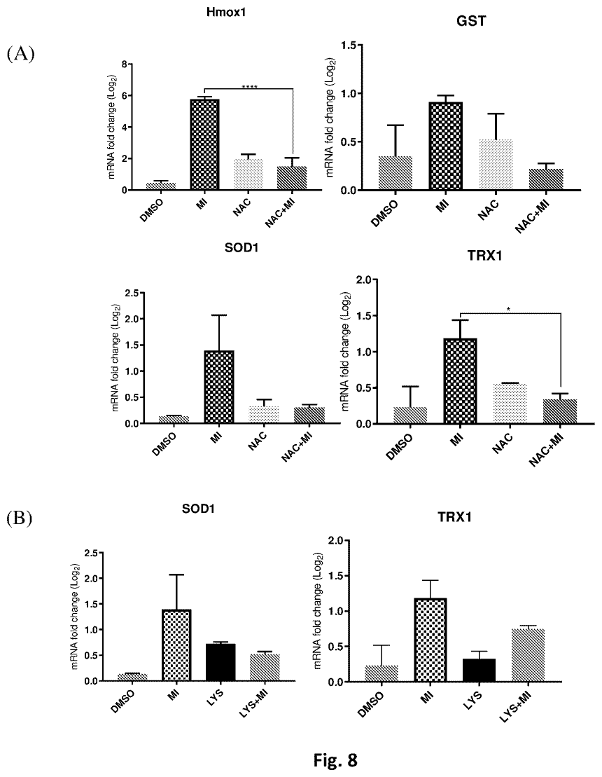

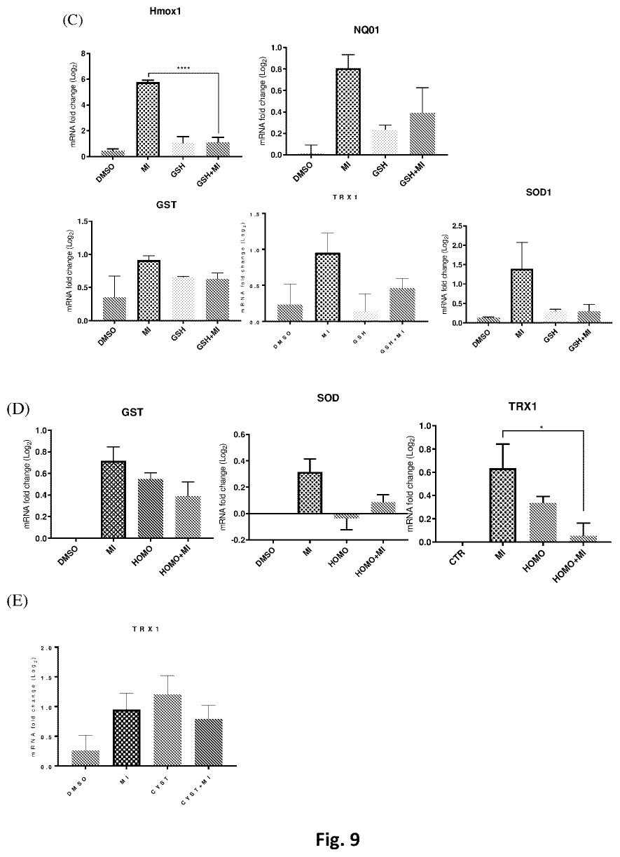

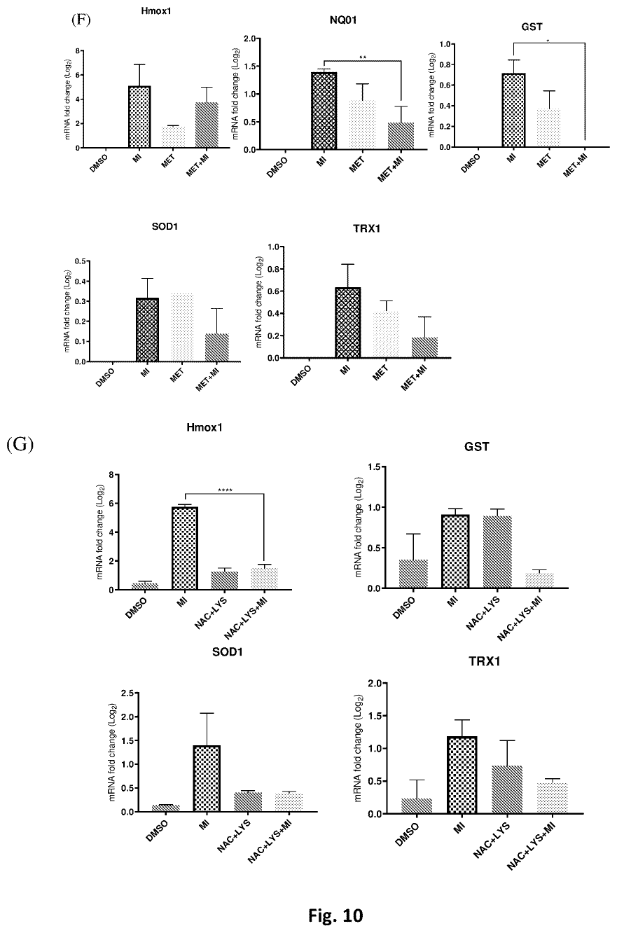

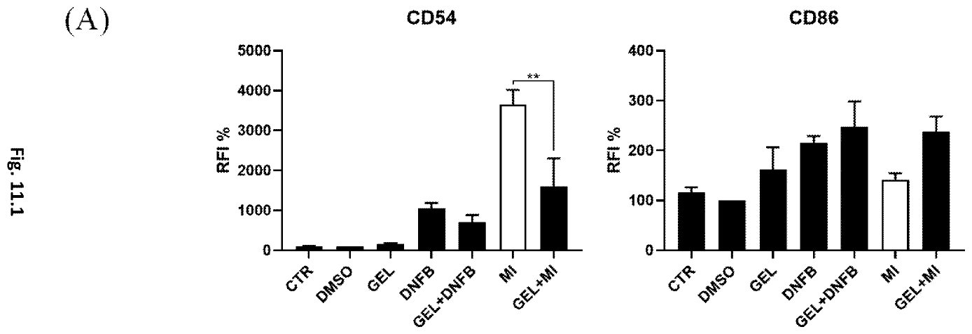

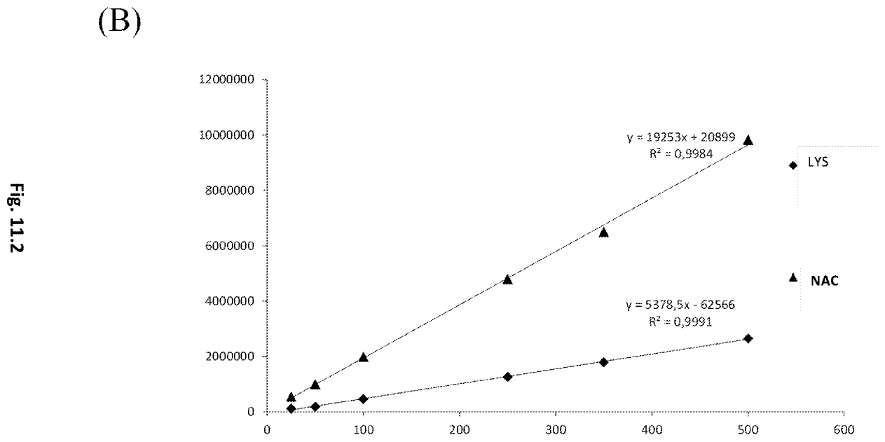

FIG. 8-10 shows the effect of NAC, LYS, NAC+LYS, GSH, HOMO and CYS on the expression of Nrf2-dependent genes in human keratinocytes, namely HMOX1, NQO1, TRX1, SOD1 and GST expression levels, after 6 h of MI incubation. In general, there is a decrease on gene expression when the cells are incubated with the active molecules before the treatment with MI, in comparison with cells treated with MI alone, particularly with a significantly decrease of HMOX1 when NAC, NAC+LYS and GSH are incubated before MI, NQO1 when, MET is incubated before MI, TRX1 when NAC and HOMO are incubated before MI and GST when MET is incubated before MI. The results demonstrate that the active molecules prevent the second key event of the development of ACD. The results are expressed as relative fold changes compared to control and represent the mean±SEM of at least 3 independent experiments. Statistical analysis was made by one-way analysis of variance (ANOVA) followed by Dunnett's multiple comparison test. * p<0.05; ** p<0.01 *** p<0.001; **** p<0.0001 significantly different compared to control.

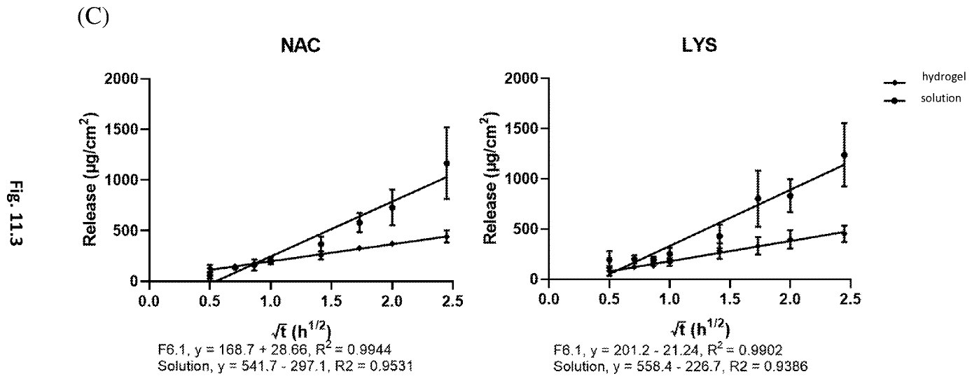

FIG. 11 shows the effect of the hydrogel containing NAC+LYS 10 mM on the maturation of THP-1 cells induced by the DNFB and MI allergens. (A) The hydrogel enriched in NAC+LYS mitigates the intensity fluorescence ratio (RFI) of co-stimulatory molecules CD54 and CD86 expression evoked by DNFB and MI. The results show the potential of the hydrogel containing NAC+LYS 10 mM in preventing the third key event of the development of ACD. The results are expressed as at least five independent experiments. * p<0.05; ** p<0.01 *** p<0.001; *: ** p<0.0001. (B) NAC and LYS calibration curves showing the quantification and the respective retention times (tr) of the molecules in suspension at 215 nm and 200 nm respectively. The following table resumes the values used to obtain the calibration curve.

| | Retention time | | Retention time | |
|---|---|---|---|---|
| Concentration | (tᵣ) | NAC | (tᵣ) | LYS |
| (mM) | (min) | (215 nm) | (min) | (200 nm) |
| 25 | 5 365 | 393398 | 2 112 | 37005 |
| 50 | 5 370 | 783683 | 2 120 | 93866 |
| 100 | 5 373 | 1449609 | 2 128 | 205742 |
| 250 | 5 383 | 3545956 | 2 125 | 656524 |
| 350 | 5 406 | 4868049 | 2 126 | 999306 |
| 500 | 5 407 | 6755103 | 2 127 | 1453986 |

FIG. 11 (C) shows the in vitro permeation test (IVPT) the experimental conditions mimic a single application of the hydrogel onto the skin and no significant quantity of the active molecules is found in the receptor compartment (below the limit of quantification (LOQ) of the HPLC method), demonstrating that neither NAC nor LYS formulated in the hydrogel can penetrate the stratum corneum of human skin, thereby remaining on the skin surface. Regarding the In vitro release testing (IVRT) profiles for NAC and LYS incorporated in the hydrogel or in solution by using Franz diffusion cells and a non-interactive synthetic membrane through time. The results show that there is a dose depletion of roughly 17% for each molecule, with a Tlag (time-lagged values) of approximately 0 h. Results are expressed as mean±SD (n=6).

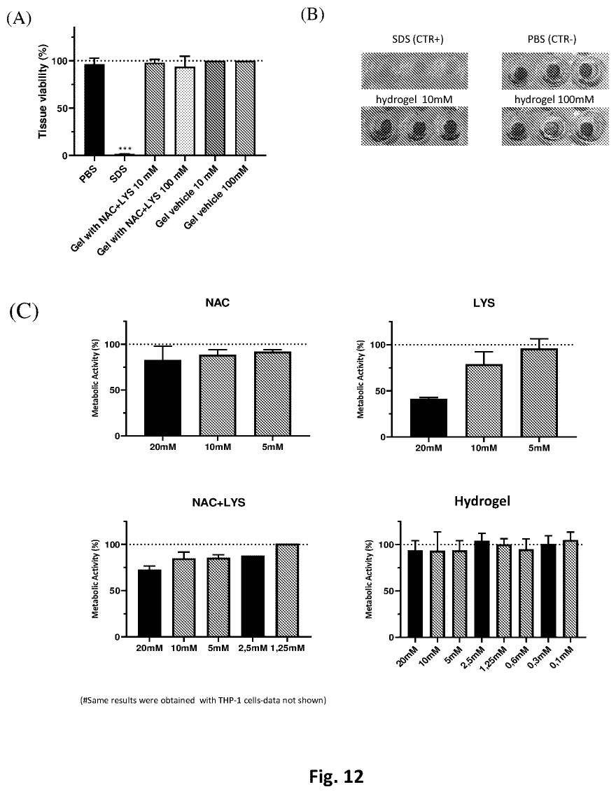

FIG. 12—shows the effect of the hydrogel enriched in NAC and LYS in SkinEthic™ Reconstructed Human Epidermis (RHE), which is an in vitro non-animal 3D model approved by OECD to evaluate the skin irritation potential of molecules. (A) RHE exposed to phosphate-buffered saline (PBS) is used as control and those treated with 5% sodium dodecyl sulfate (SDS) as the positive control. The tissue viability is assessed by the MTT assay. As expected, SDS significantly decrease the tissue viability. In contrast, any other tested conditions do not show any irritant activity, presenting tissue viability higher than 50%, which is a threshold used to classify a substance as non-irritant according to ISO 10993-10:2010. Results are expressed as a percentage (%) of the tissue viability relative to the control and represent the mean±SEM of at least three independent experiments. The following table indicates the mean and standard deviation (SD) of the tissue viability values for each condition tested.

| | Tissue viability (%) | | | SD |
|---|---|---|---|---|
| PBS | 88.82 | 110.34 | 100.84 | 10.78 |
| SDS (5%) | 1.41 | 1.44 | 1.62 | 0.11 |
| Hydrogel with | 103.05 | 88.06 | 95.48 | 7.50 |
| NAC + LYS (10 mM) | | | | |
| Hydrogel with | 97.44 | 104.41 | 114.94 | 8.81 |
| NAC + LYS (100 mM) | | | | |
| Hydrogel vehicle (10 | 115.55 | 106.41 | 100.84 | 7.43 |
| mM) | | | | |
| Hydrogel vehicle (100 | 103.46 | 105.33 | 108.31 | 2.45 |
| mM) | | | | |

FIG. 12 (C) shows images of the inserts after the MTT reduction as a final readout. (D) Metabolic activity of cells exposed to NAC, LYS, NAC+LYS and hydrogel in the keratinocyte cell line (HaCat). The results show that NAC, LYS, NAC+LYS and hydrogel do not display cytotoxicity. The statistical analysis was performed by one-way ANOVA, followed by Dunnett's multiple comparison test. **** p<0.0001: significant different compared to the control.

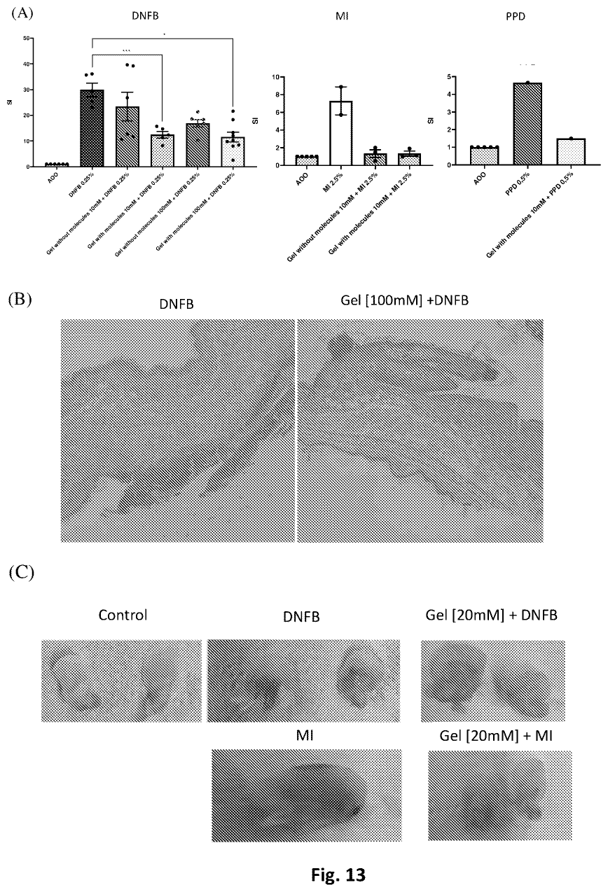

FIG. 13—(A) Stimulation index values (SI). Each substance is topically applied to the dorsal skin of the ears of mice on days 1, 2, and 3, namely the vehicle of the skin allergens (AOO); the strong allergens 2,5% DNFB, or 2,5% MI, or 0.5% PPD (positive controls); the hydrogel without molecules and the hydrogel enriched in NAC and LYS. In general, the graph shows that SI values are lower in mice treated with the hydrogel containing 10 mM or 100 mM of the active molecules before the application of the allergens, relatively to mice treated with allergens. Particularly, the SI values of DNFB when compared with SI values of hydrogel with molecules 10 mM and 100 mM+DNFB were significantly lower (B) representative histologic images of the ears, where it's possible to observe the presence of eosinophils in the ears treated with DNFB, which presence is slightly decreased in the presence of the hydrogel enriched in NAC and LYS. (C) representative histopathological macroscopic images of mice ears. It's possible to observe that DNFB and MI induce skin ulcerations, which are mitigated in the presence of the hydrogel enriched in NAC and LYS; * p<0.05; ** p<0.01; **** p<0.0001: significant different compared to the control. Acetone: olive oil (AOO), 1-Fluoro-2,4-dinitrobenzene (DNFB), Methylisothiazolinone (MI) Paraphenylenediamine (PPD).

According with the present results, it was surprisingly found that it is possible do develop a novel topical composition enriched in molecules capable of chemically sequestering skin allergens thus avoiding its interaction with skin proteins (the first adverse outcome pathway for skin sensitization) and consequently preventing the development of allergic contact dermatitis. The studies performed until now were only focused in metal allergens, chemically different from the allergens tested in this invention. Furthermore, the mechanism of action of all the ingredients listed in the literature is quite different from the mechanism of action of the molecules presented in this invention, which constitutes a innovative character of this topical composition.

For better results, a hydrogel enriched in NAC+LYS is used, providing a synergic effect between these two substances, specifically in the key events two and three. Comparing the results achieved with the molecules alone with those obtained with the concomitant use of both, it's possible to observe a synergistic effect when the two molecules were incubated simultaneously in the presence of the skin allergen DNFB, specifically for the co-stimulatory molecules CD54 and CD86. In key event 3 the synergistic effect was observed for HMOX1, SOD1 and for GST. Moreover, the viability of keratinocytes is also improved when the two molecules are combined, thus highlighting the superior safety profile of the molecules formulated in the hydrogel.

The results herein achieved are different from the prior art at least because it is the first hydrogel comprising lysin and cysteine residues aiming to prevent the development of ACD triggered by haptens reacting covalently with human skin proteins.

In an embodiment, besides this application, the molecules could also be incorporated in any other cosmetics already commercialized on the market, allowing the claim of cosmetics for ultra-sensitive or allergic skin with the capacity to prevent/rescue the development of skin allergy in individuals with some propensity to develop this pathology. The allergens sequestering molecules could also be introduced into hygiene and cleaning products enhancing the anti-allergic effect of the products already available in the market.

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof. The above described embodiments are combinable.

Where ranges are provided, the range limits are included. Furthermore, it should be understood that unless otherwise indicated or otherwise evident from the context and/or understanding of a technical expert, the values which are expressed as ranges may assume any specific value within the ranges indicated in different achievements of the invention, at one tenth of the lower limit of the interval, unless the context clearly indicates the contrary. It should also be understood that, unless otherwise indicated or otherwise evident from the context and/or understanding of a technical expert, values expressed as range may assume any sub-range within the given range, where the limits of the sub-range are expressed with the same degree of precision as the tenth of the unit of the lower limit of the range.

The following dependent claims further set out particular embodiments of the disclosure.

### REFERENCES

[1] S. HADŽAVDIĆL, P. Nives, Ž. Kristina, Š. Alen, Contact allergy: an update, G. Ital. Di DermatoloGia e Venereol. 153 (2018) 419-428. https://doi.org/10.23736/S0392-0488.17.05844-8.
[2] S. F. Martin, T. Rustemeyer, J. P. Thyssen, Recent advances in understanding and managing contact dermatitis, F1000Research 2018. 7 (2018). https://doi.org/10.12688/f1000research.13499.1.
[3] K. Wolff, R. A. Johnson, A. P. Saveedra, Fitzpatrick's Color Atlas and Synopsis of Clinical Dermatology, seventh ed, McGraw-Hill Education, 2013.
[4] M. P. Castanedo-Tardan, K. A. Zug, Fitzpatrick's Dermatology in General Medicine, 8th ed., 1998.
[5] R. S. Cvetkovski, K. J. Rothman, J. Olsen, B. Mathiesen, L. Iversen, J. D. Johansen, T. Agner, Relation between diagnoses on severity, sick leave and loss of job among patients with occupational hand eczema, Br. J. Dermatol. 152 (2005) 93-98. https://doi.org/10.1111/j.1365-2133.2005.06415.x.
[6] H. Dickel, T. Bruckner, C. Bernhard-Klimt, T. Koch, R. Scheidt, T. L. Diepgen, Surveillance scheme for occupational skin disease in the Saarland, FRG: First report from BKH-S, Contact Dermatitis. 46 (2002) 197-206. https://doi.org/10.1034/j.1600-0536.2002.460403.x.
[7] I. Kimber, R. J. Dearman, Allergic contact dermatitis: the cellular effectors, Contact Dermatitis. 46 (2002) 1-5. https://doi.org/10.1034/j.1600-0536.2002.460101.x.
[8] J. P. Thyssen, A. Linneberg, T. Menné, J. D. Johansen, The epidemiology of contact allergy in the general population-prevalence and main findings, Contact Dermatitis. 57 (2007) 287-299. https://doi.org/10.1111/j.1600-0536.2007.01220.x.
[9] J. Bourke, I. Coulson, J. English, Guidelines for the management of contact dermatitis: An update, Br. J. Dermatol. 160 (2009) 946-954. https://doi.org/10.1111/j.1365-2133.2009.09106.x.
[10] H. L. Nguyen, J. A. Yiannias, Contact Dermatitis to Medications and Skin Products, Clin. Rev. Allergy Immunol. (2018). https://doi.org/10.1007/s12016-018-8705-0.
[11] S. F. Martin, Contact dermatitis: From pathomechanisms to immunotoxicology, Exp. Dermatol. 21 (2012) 382-389. https://doi.org/10.1111/j.1600-0625.2012.01471.x.
[12] M. Peiser, T. Tralau, J. Heidler, A. M. Api, J. H. E. Arts, D. A. Basketter, J. English, T. L. Diepgen, R. C. Fuhlbrigge, A. A. Gaspari, J. D. Johansen, A. T. Karlberg, I. Kimber, J. P. Lepoittevin, M. Liebsch, H. I. Maibach, S. F. Martin, H. F. Merk, T. Platzek, T. Rustemeyer, A. Schnuch, R. J. Vandebriel, I. R. White, A. Luch, Allergic contact dermatitis: Epidemiology, molecular mechanisms, in vitro methods and regulatory aspects, Cell. Mol. Life Sci. 69 (2012) 763-781. https://doi.org/10.1007/s00018-011-0846-8.
[13] D. H. Kaplan, B. Z. Igyártó, A. A. Gaspari, Early immune events in the induction of allergic contact dermatitis, Nat. Rev. Immunol. 12 (2012) 114-124. https://doi.org/10.1038/nri3150.
[14] P. R. Esser, S. F. Martin, Pathomechanisms of Contact Sensitization, Curr. Allergy Asthma Rep. 17 (2017). https://doi.org/10.1007/s11882-017-0752-8.
[15] J. F. Lalko, I. Kimber, R. J. Dearman, G. F. Gerberick, K. Sarlo, A. M. Api, Chemical reactivity measurements: Potential for characterization of respiratory chemical allergens, Toxicol. Vitr. 25 (2011) 433-445. https://doi.org/10.1016/j.tiv.2010.11.007.
[16] I. Chipinda, J. M. Hettick, P. D. Siegel, Haptenation: Chemical Reactivity and Protein Binding, J. Allergy. 2011 (2011) 1-11. https://doi.org/10.1155/2011/839682.
[17] A. Natsch, R. Emter, H. Gfeller, T. Haupt, G. Ellis, Predicting skin sensitizer potency based on in vitro data from keratinosens and kinetic peptide binding: Global versus domain-based assessment, Toxicol. Sci. 143 (2015). https://doi.org/10.1093/toxsci/kfu229.
[18] S. F. Martin, Allergic contact dermatitis: Xenoinflammation of the skin, Curr. Opin. Immunol. 24 (2012) 720-729. https://doi.org/10.1016/j.coi.2012.08.003.
[19] E. Corsini, A. B. Engin, M. Neagu, V. Galbiati, D. Nikitovic, G. Tzanakakis, A. M. Tsatsakis, Chemical-induced contact allergy: from mechanistic understanding to risk prevention, Arch. Toxicol. 92 (2018) 3031-3050. https://doi.org/10.1007/s00204-018-2283-z.
[20] I. Kimber, A. Poole, D. A. Basketter, Skin and respiratory chemical allergy: confluence and divergence in a hybrid adverse outcome pathway, Toxicol. Res. (Camb). 7 (2018) 586-605. https://doi.org/10.1039/C7TX00272F.
[21] P. Jennings, A. Bal-Price, Methods in Pharmacology and Toxicology In Vitro Toxicology Systems, Springer Science+Business Media, New York 2014, 2014. http://www.springer.com/series/7653.
[22] W. Uter, T. Werfel, I. R. White, J. D. Johansen, Contact Allergy: A Review of Current Problems from a Clinical Perspective, Int. J. Environ. Res. Public Health. (2018). https://doi.org/10.3390/ijerph15061108.
[23] P. B. Murphy, J. N. Hooten, A. R. Atwater, Allergic Contact Dermatitis, in: NCBI Bookshelf, StatPearls Publishing, 2019: pp. 1-5.
[24] S. T. Al-Otaibi, H. A. M. Alqahtani, The Management of Contact Dermatitis, J. Dermatology Dermatologic Surg. 19 (2015) 71-73. https://doi.org/10.1142/9789812811462_0011.
[25] P. J. Nicholson, Occupational contact dermatitis: Known knowns and known unknowns, Clin. Dermatol. 29 (2011) 325-330. https://doi.org/10.1016/j.clindermatol.2010.11.012.
[26] J. Saary, R. Qureshi, V. Palda, J. DeKoven, M. Pratt, S. Skotnicki-Grant, L. Holness, A systematic review of contact dermatitis treatment and prevention, J. Am. Acad. Dermatol. 53 (2005) 845-855. https://doi.org/10.1016/j.jaad.2005.04.075.
[27] G. S. Brites, I. Ferreira, A. I. Sebastião, A. Silva, M. Carrascal, B. M. Neves, M. T. Cruz, Allergic contact dermatitis: From pathophysiology to development of new preventive strategies, Pharmacol. Res. 162 (2020). https://doi.org/10.1016/j.phrs.2020.105282.
[28] T. Lejding, M. Bruze, M. Engfeldt, M. Isaksson, C. Svedman, E. Zimerson, M. Mowitz, Barrier Creams May Not Always Perform as Expected, Dermatitis. Publish Ah (2019). https://doi.org/10.1097/der.0000000000000539.
[29] K. Wrangsio, K. Osterman, M. van Hage-Hamsten, Glove-related skin symptoms among operating theatre and dental care unit personnel, Contact Dermatitis. 30 (1994) 102-107. https://doi.org/10.1111/j.1600-0536.1994.tb00572.x.
[30] W. Wigger-Alberti, P. Elsner, Preventive measures in contact dermatitis, Clin. Dermatol. 15 (1997) 661-665. https://doi.org/10.1016/S0738-081X (97) 00001-1.
[31] C. G. T. Mathias, Prevention of occupational contact dermatitis, J. Am. Acad. Dermatol. 23 (1990) 742-748. https://doi.org/10.1016/0190-9622 (90) 70284-O.
[32] F. B. De Waard-van der Spek, K. E. Andersen, U. Darsow, C. G. Mortz, D. Orton, M. Worm, A. Muraro, P. Schmid-Grendelmeier, R. Grimalt, R. Spiewak, O. Rudzeviciene, C. Flohr, S. Halken, A. Fiocchi, L. M. Borrego, A. P. Oranje, Allergic contact dermatitis in children: which factors are relevant? (review of the literature), Pediatr. Allergy Immunol. 24 (2013) 321-329. https://doi.org/10.1111/PAI.12043.
[33] K. Osinka, A. Karczmarz, A. Krauze, W. Feleszko, Contact allergens in cosmetics used in atopic dermatitis: analysis of product composition, Contact Dermatitis. 75 (2016) 241-243. https://doi.org/10.1111/cod.12604.
[34] J. Miles, K. Mudgal, S. Patel, S. Punjabi, Contact allergy to commonly prescribed emollients, BMJ. 357 (2017). https://doi.org/10.1136/BMJ.J2008.
[35] OECD, Test No. 442E: In Vitro Skin Sensitisation, OECD, 2018. https://doi.org/10.1787/9789264264359-en.
[36] S. N. Kolle, R. Landsiedel, A. Natsch, Replacing the refinement for skin sensitization testing: Considerations to the implementation of adverse outcome pathway (AOP)-based defined approaches (DA) in OECD guidelines, Regul. Toxicol. Pharmacol. 115 (2020). https://doi.org/10.1016/J.YRTPH.2020.104713.
[37] A. Silva, M. Pereira, M. A. Carrascal, G. Brites, B. Neves, P. Moreira, R. Resende, M. M. Silva, A. E. Santos, C. Pereira, M. T. Cruz, Calcium Modulation, Anti-Oxidant and Anti-Inflammatory Effect of Skin Allergens Targeting the Nrf2 Signaling Pathway in Alzheimer's Disease Cellular Models, Int. J. Mol. Sci. 21 (2020) 7791. https://doi.org/10.3390/ijms21207791.
[38] B. Miguel Neves, S. Carvalho Rosa, trio Martins, A. Silva, M. Gonc, M. Celeste Lopes, M. Teresa Cruz, Development of an in Vitro Dendritic Cell-Based Test for Skin Sensitizer Identification, (2013). https://doi.org/10.1021/tx300472d.
[39] OECD, Test No. 439: In Vitro Skin Irritation-Reconstructed Human Epidermis Test Method, OECD, 2013. https://doi.org/10.1787/9789264203884-en.
[40] B.-I. Han, J.-S. Yi, S. J. Seo, T. S. Kim, I. Ahn, K. Ko, J. H. Kim, S. Bae, J. K. Lee, Evaluation of skin sensitization potential of chemicals by local lymph node assay using 5-bromo-2-deoxyuridine with flow cytometry, Regul. Toxicol. Pharmacol. 107 (2019) 104401. https://doi.org/10.1016/j.yrtph.2019.05.026.
[41] OECD, Test No. 442B: Skin Sensitization, OECD, 2018. https://doi.org/10.1787/9789264090996-en.

1. A method of prevention, treatment or therapy of an allergic contact dermatitis, comprising: administering to a subject an effective amount of a hydrogel comprising an allergen scavenger, wherein said allergen scavenger is:
a mixture of two amino acid selected from a list consisting of: n-acetyl cysteine, lysine, cysteine or their pharmaceutical acceptable salts; or
a mixture of two peptide comprising a terminal amino acid selected from a list consisting of: n-acetyl cysteine, lysine, cysteine or their pharmaceutical acceptable salts, or mixtures thereof.

2. The method of claim 1,
wherein the concentration of the first amino acid or peptide ranges from 0.2% to 2.0% (w/w);
wherein the concentration of the second amino acid or peptide ranges from 0.2% to 2.0% (w/w).

3. The method of claim 1, comprising n-acetyl cysteine and lysine, or their pharmaceutical acceptable salts, as allergen scavengers.

4. The method of claim 3,
wherein the concentration of n-acetyl cysteine or their pharmaceutical acceptable salt ranges from 0.2% to 2.0% (w/w);
wherein the concentration of lysine or their pharmaceutical acceptable salt ranges from 0.2% to 2.0% (w/w).

5. The method of claim 1, wherein the hydrogel is selected from a list consisting of: carbomer;
chitosan, gelatine, alginate, laminarin, hyaluronic acid, poly(vinyl alcohol), polyacrylamide, carboxymethylcellulose, sodium starch glycolate, sodium carboxymethyl starch, dextran, dextran sulfate, xanthan, gellan, pectinic acid, deoxyribonucleic acids, ribonucleic acid, albumin, polyacrolein potassium, sodium glycine carbonate, poly(acrylic acid) and its salts, polyacrylonitrile, poly(styrene sulfonate), poly(aspartic acid), polylysine, polyvinylpyrrolidone, polyvinyl alcohol, ultramylopectin, poly(ethylene glycol), neutral cellulose derivatives, microcrystalline cellulose, powdered cellulose, cellulose fibers, or starch; or combinations.

6. The method of claim 1, wherein the hydrogel further comprises keratin, collagen, glutathione, homocysteine, 2,3 dimercaptosuccinic acid, methionine, bovine serum albumin, or mixtures thereof.

7. The method of claim 1, wherein the hydrogel further comprises a chelating agent.

8. The method of claim 1, wherein the hydrogel further comprises a steroidal anti-inflammatory agent, a non-steroidal anti-inflammatory agent, a histamine H1-receptor antagonist, or mixtures thereof.

9. The method of claim 1, wherein the hydrogel comprises:
n-acetyl cysteine, in a concentration ranging from 0.2 to 2.0% (w/w), lysine, in a concentration ranging from 0.2 to 2.0% (w/w), Na₂EDTA, in a concentration ranging from 0.01 to 0.1% (w/w), benzoic acid, in a concentration equal or below 0.1% (w/w), Carbomer Interpolymer Type A, in a concentration ranging from 0.5 to 2.0% (w/w),
Isopropanol, in a concentration equal or below 30.0% (w/w), Glycerol, in a concentration equal or below 10.0% (w/w), and Water, in a concentration equal or below 70.00% (w/w).

10. A method of prevention, treatment or therapy of an allergic contact dermatitis, comprising: administering to a subject an effective amount of a topical composition, wherein said topical composition comprises:
a hydrogel comprising an allergen scavenger, wherein said allergen scavenger is:
a mixture of two amino acid selected from a list consisting of: n-acetyl cysteine, lysine, cysteine or their pharmaceutical acceptable salts; or
a mixture of two peptide comprising a terminal amino acid selected from a list consisting of: n-acetyl cysteine, lysine, cysteine or their pharmaceutical acceptable salts, or mixtures thereof;
and
acceptable pharmaceutical excipients.

11. The method of claim 10, wherein the topical composition is selected from a list consisting of: solution, serum, cream, lotion, gel, hydrogel, oil, soap, shampoo, stick- or bar-shaped solid, spray, ointment, paste, mousse or body wash.

12. (canceled)

13. The method of claim 1, wherein the allergic contact dermatitis is atopic dermatitis, eczema or seborrheic dermatitis.

14. The method of claim 10, wherein the topical composition is a skin care agent.

15. The method of claim 10, wherein the topical composition prevents or slows skin ageing.

16. The method of claim 7, wherein the chelating agent is NA₂EDTA.
